(19) United States
(12) Patent Application Publication
(10) Pub. No.: US 2026/0049108 A1
(43) Pub. Date: Feb. 19, 2026

(54) MUTANT INSECTICIDAL PROTEIN VIP3 AND ITS APPLICATION

(71) Applicant: QINGDAO KINGAGROOT SEED SCIENCE CO., LTD., Qingdao, Shandong (CN)

(72) Inventors: Lei LIAN, Qingdao, Shandong (CN); Yongchun WU, Qingdao, Shandong (CN); Bo CHEN, Qingdao, Shandong (CN); Huarong LI, Qingdao, Shandong (CN)

(21) Appl. No.: 19/103,966

(22) PCT Filed: Aug. 31, 2023
(86) PCT No.: PCT/CN2023/116207

§ 371 Date: Feb. 14, 2025

(30) Foreign Application Priority Data
Sep. 1, 2022 (CN) 202211069297.8
Mar. 9, 2023 (CN) 202310225662.8

(51) Int. Cl. C07K 14/325; C12N 15/82

(52) U.S. Cl. CPC C07K 14/325; C12N 15/8286

(57) ABSTRACT

The present invention relates to the field of biotechnology. More specifically, the present invention relates to a mutant insecticidal protein Vip3 and its application. By performing point mutation on each original Vip3 family protein sequence in the present invention, a mutant insecticidal protein Vip3 with reduced plant cytotoxicity and high expression in plant cells is obtained, which has excellent insecticidal effects on various insect pests.

### TECHNICAL FIELD

The present invention relates to the field of biotechnology. More specifically, the present invention relates to a mutant insecticidal protein Vip3 and its application.

### BACKGROUND OF THE INVENTION

Agricultural insect pests are the first crucial factor affecting crop production. With the rapid development of transgenic technology, the ability to produce insect-resistant plants by transforming Bt (Bacillus thuringiensis) insecticidal protein genes has revolutionized modern agriculture and increased the importance and value of insecticidal proteins and genes thereof. Several Bt proteins have been used in transgenic plants that produce insect resistance, including Cry1Ab protein, Cry1Ac protein, Cry1F protein, Cry2Ab protein, Cry3Bb protein, Vip3A protein, etc. However, with the popularization and application of transgenic crops, there is still an urgent need to obtain transgenic plants with high expression and effective insect resistance.

### BRIEF SUMMARY OF THE INVENTION

In order to solve the above problems existing in the prior art, the present invention provides a mutant insecticidal protein Vip3, which comprises an amino acid sequence having the following mutation(s) compared with the amino acid sequence of any Vip3 family protein: the amino acid(s) corresponding to the amino acid at position 12 in the amino acid sequence shown in SEQ ID NO: 4 being mutated into any other amino acid and/or to the amino acid at position 14 being mutated into any other amino acid.

In one specific embodiment, the mutant insecticidal protein Vip3 comprises an amino acid sequence having the following mutation(s) compared with the amino acid sequence of any Vip3 family protein: the amino acid(s) corresponding to the amino acid at position 12 in the amino acid sequence set forth in SEQ ID NO: 4 being mutated from alanine into glycine, valine, leucine, isoleucine, methionine, proline, tryptophan, serine, tyrosine, cysteine, phenylalanine, asparagine, glutamine, threonine, aspartic acid, glutamic acid, lysine, arginine or histidine; and/or to the amino acid at position 14 being mutated from proline into alanine, glycine, valine, leucine, isoleucine, methionine, tryptophan, serine, tyrosine, cysteine, phenylalanine, asparagine, glutamine, threonine, aspartic acid, glutamic acid, lysine, arginine or histidine.

In one specific embodiment, the amino acid sequence of the Vip3 family protein is as set forth in SEQ ID NO:4, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28 or SEQ ID NO: 30.

In one specific embodiment, the mutant insecticidal protein Vip3 comprises an amino acid sequence having the following mutation(s) compared with the amino acid sequence of any one Vip3 family protein:
the amino acid(s) corresponding to the amino acid at position 12 in the amino acid sequence set forth in SEQ ID NO: 4 being mutated from alanine into glycine and/or to the amino acid at position 14 being mutated from proline into glutamine;
the amino acid(s) corresponding to the amino acid at position 12 in the amino acid sequence set forth in SEQ ID NO: 4 being mutated from alanine into proline and/or to the amino acid at position 14 being mutated from proline into aspartic acid;
the amino acid(s) corresponding to the amino acid at position 12 in the amino acid sequence set forth in SEQ ID NO: 4 being mutated from alanine into glycine and/or to the amino acid at position 14 being mutated from proline into asparagine;
the amino acid(s) corresponding to the amino acid at position 12 in the amino acid sequence set forth in SEQ ID NO: 4 being mutated from alanine into glycine and/or to the amino acid at position 14 being mutated from proline into leucine;
the amino acid(s) corresponding to the amino acid at position 12 in the amino acid sequence set forth in SEQ ID NO: 4 being mutated from alanine into glycine and/or to the amino acid at position 14 being mutated from proline into arginine;
the amino acid(s) corresponding to the amino acid at position 12 in the amino acid sequence set forth in SEQ ID NO: 4 being mutated from alanine into tyrosine and/or to the amino acid at position 14 being mutated from proline into lysine;
the amino acid(s) corresponding to the amino acid at position 12 in the amino acid sequence set forth in SEQ ID NO: 4 being mutated from alanine into serine and/or to the amino acid at position 14 being mutated from proline into valine;
the amino acid(s) corresponding to the amino acid at position 12 in the amino acid sequence set forth in SEQ ID NO: 4 being mutated from alanine into histidine and/or to the amino acid at position 14 being mutated from proline into glutamine;
the amino acid(s) corresponding to the amino acid at position 12 in the amino acid sequence set forth in SEQ ID NO: 4 being mutated from alanine into histidine and/or to the amino acid at position 14 being mutated from proline into aspartic acid;
the amino acid(s) corresponding to the amino acid at position 12 in the amino acid sequence set forth in SEQ ID NO: 4 being mutated from alanine into valine and/or to the amino acid at position 14 being mutated from proline into cysteine;
the amino acid(s) corresponding to the amino acid at position 12 in the amino acid sequence set forth in SEQ ID NO: 4 being mutated from alanine into proline and/or to the amino acid at position 14 being mutated from proline into glycine;
the amino acid(s) corresponding to the amino acid at position 12 in the amino acid sequence set forth in SEQ ID NO: 4 being mutated from alanine into tryptophan and/or to the amino acid at position 14 being mutated from proline into threonine;
the amino acid(s) corresponding to the amino acid at position 12 in the amino acid sequence set forth in SEQ ID NO: 4 being mutated from alanine into glycine and/or to the amino acid at position 14 being mutated from proline into aspartic acid;
the amino acid(s) corresponding to the amino acid at position 12 in the amino acid sequence set forth in SEQ ID NO: 4 being mutated from alanine into phenylalanine and/or to the amino acid at position 14 being mutated from proline into isoleucine;
the amino acid(s) corresponding to the amino acid at position 12 in the amino acid sequence set forth in SEQ ID NO: 4 being mutated from alanine into glycine and/or to the amino acid at position 14 being mutated from proline into alanine;
the amino acid(s) corresponding to the amino acid at position 12 in the amino acid sequence set forth in SEQ ID NO: 4 being mutated from alanine into phenylalanine and/or to the amino acid at position 14 being mutated from proline into histidine;
the amino acid(s) corresponding to the amino acid at position 12 in the amino acid sequence set forth in SEQ ID NO: 4 being mutated from alanine into asparagine and/or to the amino acid at position 14 being mutated from proline into glutamine;
the amino acid(s) corresponding to the amino acid at position 12 in the amino acid sequence set forth in SEQ ID NO: 4 being mutated from alanine into glutamine and/or to the amino acid at position 14 being mutated from proline into histidine;
the amino acid(s) corresponding to the amino acid at position 12 in the amino acid sequence set forth in SEQ ID NO: 4 being mutated from alanine into proline and/or to the amino acid at position 14 being mutated from proline into methionine;
the amino acid(s) corresponding to the amino acid at position 12 in the amino acid sequence set forth in SEQ ID NO: 4 being mutated from alanine into aspartic acid and/or to the amino acid at position 14 being mutated from proline into phenylalanine;
the amino acid(s) corresponding to the amino acid at position 12 in the amino acid sequence set forth in SEQ ID NO: 4 being mutated from alanine into histidine and/or to the amino acid at position 14 being mutated from proline into lysine;
the amino acid(s) corresponding to the amino acid at position 12 in the amino acid sequence set forth in SEQ ID NO: 4 being mutated from alanine into phenylalanine and/or to the amino acid at position 14 being mutated from proline into methionine;
the amino acid corresponding to the amino acid at position 12 in the amino acid sequence set forth in SEQ ID NO: 4 being mutated from alanine into glutamic acid;
the amino acid(s) corresponding to the amino acid at position 12 in the amino acid sequence set forth in SEQ ID NO: 4 being mutated from alanine into glycine and/or to the amino acid at position 14 being mutated from proline into isoleucine;
the amino acid(s) corresponding to the amino acid at position 12 in the amino acid sequence set forth in SEQ ID NO: 4 being mutated from alanine into aspartic acid and/or to the amino acid at position 14 being mutated from proline into glycine;
the amino acid(s) corresponding to the amino acid at position 12 in the amino acid sequence set forth in SEQ ID NO: 4 being mutated from alanine into glutamine and/or to the amino acid at position 14 being mutated from proline into valine;
the amino acid(s) corresponding to the amino acid at position 12 in the amino acid sequence set forth in SEQ ID NO: 4 being mutated from alanine into serine and/or to the amino acid at position 14 being mutated from proline into glycine;
the amino acid(s) corresponding to the amino acid at position 12 in the amino acid sequence set forth in SEQ ID NO: 4 being mutated from alanine into phenylalanine and/or to the amino acid at position 14 being mutated from proline into glycine;
the amino acid(s) corresponding to the amino acid at position 12 in the amino acid sequence set forth in SEQ ID NO: 4 being mutated from alanine into glycine and/or to the amino acid at position 14 being mutated from proline into serine;
the amino acid(s) corresponding to the amino acid at position 12 in the amino acid sequence set forth in SEQ ID NO: 4 being mutated from alanine into arginine and/or to the amino acid at position 14 being mutated from proline into methionine;
the amino acid corresponding to the amino acid at position 12 in the amino acid sequence set forth in SEQ ID NO: 4 being mutated from alanine into histidine;
the amino acid corresponding to the amino acid at position 12 in the amino acid sequence set forth in SEQ ID NO: 4 being mutated from alanine into threonine;
the amino acid corresponding to the amino acid at position 14 in the amino acid sequence set forth in SEQ ID NO: 4 being mutated from proline into cysteine;
the amino acid corresponding to the amino acid at position 14 in the amino acid sequence set forth in SEQ ID NO: 4 being mutated from proline into valine;
the amino acid corresponding to the amino acid at position 14 in the amino acid sequence set forth in SEQ ID NO: 4 being mutated from proline into alanine;
the amino acid corresponding to the amino acid at position 14 in the amino acid sequence set forth in SEQ ID NO: 4 being mutated from proline into aspartic acid;
the amino acid corresponding to the amino acid at position 14 in the amino acid sequence set forth in SEQ ID NO: 4 being mutated from proline into glutamic acid;
the amino acid corresponding to the amino acid at position 14 in the amino acid sequence set forth in SEQ ID NO: 4 being mutated from proline into phenylalanine;
the amino acid corresponding to the amino acid at position 14 in the amino acid sequence set forth in SEQ ID NO: 4 being mutated from proline into histidine;
the amino acid corresponding to the amino acid at position 14 in the amino acid sequence set forth in SEQ ID NO: 4 being mutated from proline into isoleucine; or, the amino acid corresponding to the amino acid at position 14 in the amino acid sequence set forth in SEQ ID NO: 4 being mutated from proline into lysine.

In another specific embodiment, the amino acid sequence of the mutant insecticidal protein Vip3 is as set forth in SEQ ID NO:1, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29 or SEQ ID NO: 41-80.

The present invention also provides an isolated polynucleotide which comprises a nucleic acid sequence encoding the mutant insecticidal protein Vip3 or a complementary sequence thereof.

In one specific embodiment, the polynucleotide is DNA, RNA, or a hybrid thereof.

In one specific embodiment, the polynucleotide is single-stranded or double-stranded.

In one specific embodiment, the polynucleotide has a nucleic acid sequence selected from:
(1) a nucleic acid sequence encoding the amino acid sequence as shown in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29 or SEQ ID NO: 41-80, or a complementary sequence thereof;
(2) the nucleic acid sequence as shown in any one of SEQ ID NO: 2, SEQ ID NO: 31-40, SEQ ID NO: 81-120 or a complementary sequence thereof;
(3) a nucleic acid sequence that hybridizes to the sequence shown in (1) or (2) under stringent conditions; and/or
(4) a nucleic acid sequence encoding the same amino acid sequence as the sequence shown in (1) or (2) due to degeneracy of the genetic code, or a complementary sequence thereof.

In another specific embodiment, the nucleic acid sequence is optimized for expression in plant cells.

The present invention also provides an expression vector comprising the polynucleotide and an expression regulatory element operably linked thereto.

The present invention also provides an expression vector comprising gene tandem expression cassettes that express the mutant insecticidal protein Vip3 and Pat.

In one specific embodiment, the nucleotide sequence of the genetically mutant insecticidal protein Vip3 is shown in any one of SEQ ID NO: 2, SEQ ID NO: 31-40, SEQ ID NO: 81-120 and the nucleotide sequence of the pat gene is SEQ ID NO: 6.

In another specific embodiment, the gene tandem expression cassettes also comprise:
a CaMV 35S promoter which initiates the expression of the pat and of which the nucleotide sequence is as shown in SEQ ID NO: 5, and a CaMV poly (A) signal termination sequence which terminates the expression of the gene and of which the nucleotide sequence is as shown in SEQ ID NO: 7;
an OsUbi2 promoter which initiates the expression of the mutant insecticidal protein Vip3 and of which the nucleotide sequence is as shown in SEQ ID NO: 8, a chloroplast leading peptide CTP-TS-SSU whose nucleotide sequence is as shown in SEQ ID NO: 3, and a T-Ara5 terminator which terminates the expression of the gene and of which the nucleotide sequence is as shown in SEQ ID NO: 9.

In another specific embodiment, the nucleotide sequence of the expression vector is as shown in SEQ ID NO: 10.

The present invention also provides a host cell comprising the polynucleotide or the expression vector.

In one specific embodiment, the host cell is a plant cell.

The present invention also provides a method of cultivating a transgenic plant having or with enhanced insect resistance and a plant produced by the method, which includes regenerating the plant cell into a plant.

The present invention also provides an application of the expression vector or the host cell in improving insect-resistant characteristics of a plant, preparing an agent with insect-resistant effect, or cultivating a transgenic plant having or with enhanced insect resistance.

The present invention also provides a method of managing insect resistance or controlling an insect, which comprises contacting the insect with at least the above-mentioned plant, wherein the insect contacts at least the mutant insecticidal protein Vip3 by ingesting tissues of the plant, after which the insect is inhibited in growth and/or died, thereby achieving management of resistance against the insect or achieving control of the insect damaging the plant.

In one specific embodiment, the plant is maize, cotton, or soybean.

In one specific embodiment, the “insect-resistant” is lepidopteran-resistant or the insect is a lepidopteran.

By performing point mutation on the original Vip3 family protein sequence in the present invention, a mutant Vip3 family insecticidal protein with reduced plant cytotoxicity and high expression in plant cells is obtained, which has excellent insecticidal effects on various insect pests.

### BRIEF DESCRIPTION OF THE SEVERAL VIEWS OF THE DRAWING(S)

FIG. 1 shows the schematic of the pQYI0187 vector.

FIG. 2 shows the comparison of plant cytotoxicity of transgenic maizes QYI186 (upper half: transformed with MIR162 Vip3Aa) and QYI187 (lower half: transformed with Vip3 Aa-K1) 4 weeks after callus differentiation.

FIG. 3 shows the comparison of the phytotoxicity of the other Vip3 Aa protein mutants.

FIG. 4 shows the death rates of Spodoptera frugiperda larvae using different multiple diluents of frozen leaf powders of transgenic maizes QYI187 and QYI186. The image on the left shows QYI187 diluted 50 times and the image on the right shows QYI186 diluted 4 times.

FIG. 5 shows the representative experimental results of feeding Helicoverpa armigera with non-transgenic and transgenic soybean leaves. The image on the left shows the leaf of a non-transgenic wild-type recipient soybean, and the image on the right shows the leaf of a transgenic soybean transformed with Vip3Aa-K1.

### DETAILED DESCRIPTION OF THE INVENTION

Some terms used in this specification are defined as follows.

In the present invention, the “plant” should be understood as any differentiated multicellular organism capable of performing photosynthesis, in particular monocotyledonous or dicotyledonous plants.

In the present invention, the term “plant tissue” or “plant part” includes a plant cell, protoplast, plant tissue culture, plant callus, plant piece as well as a plant embryo, pollen, ovule, seed, leaf, stem, flower, branch, shoot, fruit, pit, ears, root, root tip, anther, etc.

In the present invention, the “plant cell” should be understood as any cell derived or found in a plant, which is capable of forming, for example, undifferentiated tissues such as calli, differentiated tissues such as embryos, constituent parts of a plant, plants, or seeds.

In the present invention, the “host organism” should be understood as any mono- or multi-cellular organism into which a nucleic acid encoding a mutant protein can be introduced, including, for example, bacteria such as Escherichia coli, fungi such as yeasts (e.g., Saccharomyces cerevisiae), molds (e.g., Aspergillus), plant cells, plants, and the like.

The terms “protein”, “polypeptide” and “peptide” can be used interchangeably in the present invention and refer to a polymer of amino acid residues, including a polymer of chemical analogs in which one or more amino acid residues are natural amino acid residues. The proteins and polypeptides of the present invention may be recombinantly produced or chemically synthesized.

The specific amino acid positions (numbering) in the protein of the present invention are determined by aligning the amino acid sequence of a target protein with Vip3 Aa using standard sequence alignment tools. For example, two sequences are aligned using the Smith-Waterman algorithm or the ClustalW2 algorithm, wherein the sequences are considered aligned when the alignment score is the highest. Alignment scores can be calculated according to the method described in Wilbur, W. J. and Lipman, D. J. (1983) Rapid similarity searches of nucleic acid and protein data banks. Proc. Natl. Acad. Sci. USA, 80:726-730. Default parameters are preferably used in the ClustalW2 (1.82) algorithm: Protein gap opening penalty=10.0; Protein gap extension penalty=0.2; Protein weight matrix=Gonnet; Protein/DNA endgap=−1; Protein/DNA GAPDIST=4.

The AlignX program (a part of the Vector NTI WorkGroup) is preferably adopted to accommodate default parameters of multiple alignment (Gap opening penalty: 10; Gap extension penalty: 0.05). The position of a specific amino acid in the protein of the present invention is determined by aligning the amino acid sequence of the protein with Vip3Aa.

Amino acid sequence identity can be determined by conventional methods using the BLAST algorithm (Altschul et al., 1990, Mol. Biol. 215:403-10) obtained from the National Center for Biotechnology Information, USA (www.ncbi.nim.nih.gov/) with default parameters.

It is also clear to those skilled in the art that the structure of a protein may be altered without adversely affecting its activity and functionality. For example, one or more conservative amino acid substitutions can be introduced into the amino acid sequence of a protein without adversely affecting the activity and/or three-dimensional configuration of the protein molecule. Examples and embodiments of the conservative amino acid substitutions are apparent to those skilled in the art. Specifically, an amino acid residue can be substituted with another amino acid residue that belongs to the same group as the site to be substituted does, that is, using a non-polar amino acid residue to substitute another non-polar amino acid residue, using an uncharged polar amino acid residue to substitute another uncharged polar amino acid, using an alkaline amino acid residue to substitute another alkaline amino acid residue, and using an acidic amino acid residue to substitute another acidic amino acid residue. Conservative substitutions that one amino acid is substituted with another amino acid of the same group are within the scope of the present invention as long as the substitutions do not impair the biological activity of the protein.

Therefore, in addition to the above-mentioned mutations, the mutant protein of the present invention may further comprise one or more other mutations, such as conservative substitutions, in the amino acid sequence. Moreover, the present invention encompasses a mutant protein that also comprises one or more other non-conservative substitutions, as long as the non-conservative substitutions do not significantly affect the desired functions or biological activity of the protein of the present invention.

As well known in the art, one or more amino acid residues can be deleted from the N- and/or C-terminus of a protein, and the protein still retains its functional activity. Thus, in another aspect, the present invention also relates to fragments that have one or more amino acid residues deleted from N- and/or C-terminus of mutant proteins while retain their desired functional activity, which are also within the scope of the present invention and called bioactive fragments. In the present invention, the “bioactive fragment” refers to a portion of the mutant protein of the present invention which retains biological activity of the mutant protein of the present invention. For example, a bioactive fragment of a mutant protein may be a portion that has one or more (e.g., 1-50, 1-25, 1-10 or 1-5; e.g., 1, 2, 3, 4 or 5) amino acid residues deleted from the N- and/or C-terminus of the protein while still retains biological activity of its full-length protein.

The terms “Vip3 family protein”, “Vip3 family gene”, and “Vip3” are the results of the classification of Vip (vegetative insecticidal protein) proteins based on the homology of amino acid sequences, including vip3A, vip3B, vip3C and other genes.

In one specific embodiment, the amino acid sequence of the Vip3 family protein is as set forth in SEQ ID NO:4, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28 or SEQ ID NO: 30.

The term “mutation” refers to a single amino acid variation in a polypeptide and/or at least a single nucleotide variation in a nucleic acid sequence relative to a canonical sequence, wild-type sequence, or reference sequence.

The terms “polynucleotide”, “nucleic acid”, “nucleic acid molecule” or “nucleic acid sequence” can be used interchangeably, referring to an oligonucleotide, nucleotide or polynucleotide and a fragment or part thereof, which may be single-stranded or double-stranded, representing a sense or antisense strand. A nucleic acid includes DNA, RNA, or a hybrid thereof, and can be of natural or synthetic origin. For example, a nucleic acid may include a mRNA or cDNA. A nucleic acid may include a nucleic acid that has been amplified (e.g., by the polymerase chain reaction). The nucleotide designation “R” means purine such as guanine or adenine; “Y” means pyrimidine such as cytosine or thymine (uracil in the case of RNA); “M” means adenine or cytosine; “K” means guanine or thymine; and “W” means adenine or thymine.

The term “isolated”, when referring to a nucleic acid, means a nucleic acid that is apart from a substantial portion of the genome in which it naturally occurs and/or is substantially separated from other cellular components which naturally accompany the nucleic acid. For example, any nucleic acid that has been produced synthetically (e.g., by serial base condensation) is considered to be isolated. Likewise, a nucleic acid that is recombinantly expressed, or cloned, or produced by a primer extension reaction (e.g., PCR), or otherwise excised from a genome is also considered to be isolated.

It will be apparent to those skilled in the art that a variety of different nucleic acid sequences can encode the amino acid sequence disclosed herein due to degeneracy of the genetic code. It is within the ability of one of ordinary skill in the art to generate other nucleic acid sequences encoding a same protein, and thus the present invention encompasses nucleic acid sequences that encode the same amino acid sequence due to degeneracy of the genetic code. For example, in order to achieve high expression of a heterologous gene in a target host organism, such as a plant, the gene can be optimized using host-preferred codons for better expression.

The term “transgenic” plant refers to a plant comprising a heterologous polynucleotide. Preferably, a heterologous polynucleotide is stably integrated into a genome, allowing the polynucleotide to be passed on to successive generations. A heterologous polynucleotide can be integrated into a genome alone or as a part of a recombinant expression cassette. The “transgenic” is used herein to refer to any cell, cell line, callus, tissue, plant part or plant whose genotype is altered due to the presence of a heterologous nucleic acid, including those originally altered transgenic organisms or cells, as well as those resulting from crossing or asexual propagation of originally altered transgenic organisms or cells. The term “transgenic” as used herein is not intended to include altering genomes (chromosomal or extrachromosomal) by conventional plant breeding methods (e.g., crossing) or by naturally occurring events (e.g., self-fertilization, random cross-fertilization, non-recombinant viral infection, non-recombinant bacterial transformation, non-recombinant transposition, or spontaneous mutation).

The herbicide-resistant pat gene and the Vip3 family gene of the present invention may be introduced into plants according to methods commonly used in the art, by transgenic operations via appropriate plant transformation expression vectors.

Selection of any appropriate promoter is a common practice in the art when performing genetic modification, including vectors, in plants. For example, promoters commonly used in genetic modification in plants include, but are not limited to, the SP6 promoter, T7 promoter, T3 promoter, PM promoter, maize ubiquitin promoter, cauliflower mosaic virus (CaMV) 35S promoter, nopaline synthase (nos) promoter, figwort mosaic virus 35S promoter, sugarcane bacilliform virus promoter, commelina yellow mottle virus promoter, light inducible promoter from the ribulose-1,5-ketose carboxylase (ssRUBISCO small subunit), rice cytoplast triose-phosphate isomerase (TPI) promoter, Arabidopsis adenine phosphoribosyl transferase (APRT) promoter, octopine synthase promoter and BCB (blue copper-binding protein) promoter.

A plant transgenic vector comprises a polyadenylation signal sequence causing the 3′-terminal polyadenylation. Examples include, but are not limited to, NOS 3′-terminal derivatives of the nopaline synthase gene of Agrobacterium, octopine synthase 3′-terminal derivatives of the octopine synthase gene of Agrobacterium, 3′-terminus of the tomato or potato proteinase inhibitor I or II gene, CaMV Poly A signal sequence, 3′-terminus of the rice α-amylase gene and 3′-terminus of the phaseoline gene.

A vector also comprises the coding gene of a selectable marker that is a reporter molecule, and examples of selectable markers include, but are not limited to, antibiotics (e.g., neomycin, carbenicillin, kanamycin, spectinomycin, hygromycin, bleomycin, chloramphenicol, etc.) or herbicide-resistant (glyphosate, glufosinate-ammonium, glufosinate, etc.) genes.

Methods of vector transformation include introducing recombinant plasmids into plants using methods such as Agrobacterium-mediated transformation, electroporation, microparticle bombardment, polyethylene glycol (PEG)-medium absorption, etc.

The recipients of plant transformation in the present invention include plant cells (including suspension culture cells), protoplasts, calli, hypocotyls, seeds, cotyledons, shoots, and mature plant bodies.

The scope of transgenic plants includes not only the plant body obtained at the time of gene introduction, but also clones and progeny thereof (T1 generation, T2 generation or subsequent generations). The scope of the present invention also includes all mutants and variants, by crossing and fusion of the above-mentioned transgenic plants, exhibiting the characteristics of the first-generation transgenic plants. The scope of the present invention also includes plant parts, such as seed, flower, stem, fruit, leaf, root, tuber, tuberous stem, which are derived from plants that have been genetically modified in advance by the methods mentioned in the present invention or from progeny thereof, and at least consist of a portion of transgenically modified cells.

The “insecticidal” or “insect-resistant” in the present invention refers to being toxic to agricultural crop pests, so as to “control” and/or “prevent” crop insect pests. Preferably, the “insecticidal” or “insect-resistant” refers to killing crop insect pests. The above-mentioned insect pests include lepidopterans, e.g., Ostrinia nubilalis and/or Spodoptera frugiperda.

The “insects being inhibited in growth” in the present invention refers to being sublethal, that is, not yet lethal but can cause certain effects on growth and development, behaviour, physiology, biochemistry, tissues, and the like, such as delay and/or cessation of growth and development. Meanwhile, plants should be morphologically normal and can be cultivated using conventional methods for consumption and/or production of products.

The present invention may be implemented in a variety of different forms and implementation methods should not be limited to those set forth herein. The embodiments herein are provided to achieve a thorough and complete disclosure, allowing those skilled in the art to fully understand the scope of the invention. The same reference numbers refer to the same elements in the present invention.

The terminology used herein is for the purpose of describing particular embodiments rather than setting limitations. Unless otherwise specified expressly in the context, “a”, “an”, and “the” used in the Chinese and English versions of the above content also include their plural forms. The terms “comprises” and/or “comprising”, or “includes” and/or “including” used herein specifically refer to the presence of features, factors and/or components described herein, and do not exclude the presence and addition of one or more other features, factors and/or components. The term “and/or” used in the above content includes all items in one or more combined lists.

The present invention has been illustrated in detail by a series of embodiments, however, the present invention is not limited to the disclosed embodiments. Any numerical changes, substitutions, replacements and so on, which are within the scope of the present invention, are not stated herein, or may be modified according to public needs.

DESCRIPTION OF SEQUENCES

| | | Associated |
|---|---|---|
| Sequence No. | Gene Information | Vector |
| SEQ ID NO: 1 | Amino acid sequence of Vip3Aa-K1 | pQYI0187 |
| SEQ ID NO: 2 | Nucleotide sequence of maize-codon | |
| | optimized Vip3Aa-K1 | |
| SEQ ID NO: 3 | Chloroplast localized peptide | |
| | CTP-TS-SSU | |
| SEQ ID NO: 4 | Amino acid sequence of MIR162 Vip3Aa | pQYI0186 |
| SEQ ID NO: 5 | CaMV 35S promoter | |
| SEQ ID NO: 6 | Pat | |
| SEQ ID NO: 7 | CaMV poly (A) signal | |
| SEQ ID NO: 8 | OsUbi2 promoter | |
| SEQ ID NO: 9 | T-Ara5 | |
| SEQ ID NO: 10 | Full-length pQYI0187 vector | |
| SEQ ID NO: 11 | Double mutant of Vip3Aa truncated | pQYI0011 |
| | protein | |
| SEQ ID NO: 12 | Amino acid sequence of Vip3Aa truncated | pQYI0012 |
| | protein | |
| SEQ ID NO: 13 | Double mutant of Vip3Aa19 | pQYI0013 |
| SEQ ID NO: 14 | Amino acid sequence of Vip3Aa19 | pQYI0014 |
| SEQ ID NO: 15 | Double mutant of Vip3Aa42 | pQYI0015 |
| SEQ ID NO: 16 | Amino acid sequence of Vip3Aa42 | pQYI0016 |
| SEQ ID NO: 17 | Double mutant of Vip3AfAa | pQYI0017 |
| SEQ ID NO: 18 | Amino acid sequence of Vip3AfAa | pQYI0018 |
| SEQ ID NO: 19 | Double mutant of Vip3Aa39 | pQYI0019 |
| SEQ ID NO: 20 | Amino acid sequence of Vip3Aa39 | pQYI0020 |
| SEQ ID NO: 21 | Double mutant of unnamed amino acid | pQYI0021 |
| | sequence of Vip3 family protein | |
| SEQ ID NO: 22 | Unnamed amino acid sequence of Vip3 | pQYI0022 |
| | family protein | |
| SEQ ID NO: 23 | Double mutant of heterozygous Vip3A-C | pQYI0023 |
| | toxin | |
| SEQ ID NO: 24 | Amino acid sequence of heterozygous | pQYI0024 |
| | Vip3A-C toxin | |
| SEQ ID NO: 25 | Double mutant of modified Vip3A | pQYI0025 |
| | polypeptide | |
| SEQ ID NO: 26 | Amino acid sequence of modified Vip3A | pQYI0026 |
| | polypeptide | |
| SEQ ID NO: 27 | Double mutant of Vip3Ah1 | pQYI0027 |
| SEQ ID NO: 28 | Amino acid sequence of Vip3Ah1 | pQYI0028 |
| SEQ ID NO: 29 | Double mutant of Vip3Ca2 | pQYI0029 |
| SEQ ID NO: 30 | Amino acid sequence of Vip3Ca2 | pQYI0030 |
| SEQ ID NO: 31 | Nucleotide sequence of double mutant of | pQYI0011 |
| | Vip3Aa truncated protein | |
| SEQ ID NO: 32 | Nucleotide sequence of double mutant of | pQYI0013 |
| | Vip3Aa19 protein | |
| SEQ ID NO: 33 | Nucleotide sequence of double mutant of | pQYI0015 |
| | Vip3Aa42 protein | |
| SEQ ID NO: 34 | Nucleotide sequence of double mutant of | pQYI0017 |
| | Vip3AfAa protein | |
| SEQ ID NO: 35 | Nucleotide sequence of double mutant of | pQYI0019 |
| | Vip3Aa39 protein | |
| SEQ ID NO: 36 | Nucleotide sequence of double mutant | pQYI0021 |
| | of unnamed protein of Vip3 family | |
| SEQ ID NO: 37 | Nucleotide sequence of double mutant of | pQYI0023 |
| | heterozygous Vip3A-C toxin | |
| SEQ ID NO: 38 | Nucleotide sequence of double mutant of | pQYI0025 |
| | modified Vip3A polypeptide | |
| SEQ ID NO: 39 | Nucleotide sequence of double mutant of | pQYI0027 |
| | Vip3Ah1 protein | |
| SEQ ID NO: 40 | Nucleotide sequence of double mutant of | pQYI0029 |
| | Vip3Ca2 protein | |
| SEQ ID NO: 41 | Amino acid sequence of mVip3Aa | |
| | (A12P + P14D) | |
| SEQ ID NO: 42 | Amino acid sequence of mVip3Aa | |
| | (A12G + P14N) | |
| SEQ ID NO: 43 | Amino acid sequence of mVip3Aa | |
| | (A12G + P14L) | |
| SEQ ID NO: 44 | Amino acid sequence of mVip3Aa | |
| | (A12G + P14R) | |
| SEQ ID NO: 45 | Amino acid sequence of mVip3Aa | |
| | (A12Y + P14K) | |
| SEQ ID NO: 46 | Amino acid sequence of mVip3Aa | |
| | (A12S + P14V) | |
| SEQ ID NO: 47 | Amino acid sequence of mVip3Aa | |
| | (A12H + P14Q) | |
| SEQ ID NO: 48 | Amino acid sequence of mVip3Aa | |
| | (A12H + P14D) | |
| SEQ ID NO: 49 | Amino acid sequence of mVip3Aa | |
| | (A12V + P14C) | |
| SEQ ID NO: 50 | Amino acid sequence of mVip3Aa | |
| | (A12P + P14G) | |
| SEQ ID NO: 51 | Amino acid sequence of mVip3Aa | |
| | (A12W + P14T) | |
| SEQ ID NO: 52 | Amino acid sequence of mVip3Aa | |
| | (A12G + P14D) | |
| SEQ ID NO: 53 | Amino acid sequence of mVip3Aa | |
| | (A12F + P14I) | |
| SEQ ID NO: 54 | Amino acid sequence of mVip3Aa | |
| | (A12G + P14A) | |
| SEQ ID NO: 55 | Amino acid sequence of mVip3Aa | |
| | (A12F + P14H) | |
| SEQ ID NO: 56 | Amino acid sequence of mVip3Aa | |
| | (A12N + P14Q) | |
| SEQ ID NO: 57 | Amino acid sequence of mVip3Aa | |
| | (A12Q + P14H) | |
| SEQ ID NO: 58 | Amino acid sequence of mVip3Aa | |
| | (A12P + P14M) | |
| SEQ ID NO: 59 | Amino acid sequence of mVip3Aa | |
| | (A12D + P14F) | |
| SEQ ID NO: 60 | Amino acid sequence of mVip3Aa | |
| | (A12H + P14K) | |
| SEQ ID NO: 61 | Amino acid sequence of mVip3Aa | |
| | (A12F + P14M) | |
| SEQ ID NO: 62 | Amino acid sequence of mVip3Aa | |
| | (A12E) | |
| SEQ ID NO: 63 | Amino acid sequence of mVip3Aa | |
| | (A12G + P14I) | |
| SEQ ID NO: 64 | Amino acid sequence of mVip3Aa | |
| | (A12D + P14G) | |
| SEQ ID NO: 65 | Amino acid sequence of mVip3Aa | |
| | (A12Q + P14V) | |
| SEQ ID NO: 66 | Amino acid sequence of mVip3Aa | |
| | (A12S + P14G) | |
| SEQ ID NO: 67 | Amino acid sequence of mVip3Aa | |
| | (A12F + P14G) | |
| SEQ ID NO: 68 | Amino acid sequence of mVip3Aa | |
| | (A12G + P14S) | |
| SEQ ID NO: 69 | Amino acid sequence of mVip3Aa | |
| | (A12R + P14M) | |
| SEQ ID NO: 70 | Amino acid sequence of mVip3Aa | |
| | (A12H) | |
| SEQ ID NO: 71 | Amino acid sequence of mVip3Aa | |
| | (A12T) | |
| SEQ ID NO: 72 | Amino acid sequence of mVip3Aa | |
| | (P14C) | |
| SEQ ID NO: 73 | Amino acid sequence of mVip3Aa | |
| | (P14V) | |
| SEQ ID NO: 74 | Amino acid sequence of mVip3Aa | |
| | (P14A) | |
| SEQ ID NO: 75 | Amino acid sequence of mVip3Aa | |
| | (P14D) | |
| SEQ ID NO: 76 | Amino acid sequence of mVip3Aa | |
| | (P14E) | |
| SEQ ID NO: 77 | Amino acid sequence of mVip3Aa | |
| | (P14F) | |
| SEQ ID NO: 78 | Amino acid sequence of mVip3Aa | |
| | (P14H) | |
| SEQ ID NO: 79 | Amino acid sequence of mVip3Aa | |
| | (P14I) | |
| SEQ ID NO: 80 | Amino acid sequence of mVip3Aa | |
| | (P14K) | |
| SEQ ID NO: 81 | Nucleotide sequence of mVip3Aa | |
| | (A12P + P14D) | |
| SEQ ID NO: 82 | Nucleotide sequence of mVip3Aa | |
| | (A12G + P14N) | |
| SEQ ID NO: 83 | Nucleotide sequence of mVip3Aa | |
| | (A12G + P14L) | |
| SEQ ID NO: 84 | Nucleotide sequence of mVip3Aa | |
| | (A12G + P14R) | |
| SEQ ID NO: 85 | Nucleotide sequence of mVip3Aa | |
| | (A12Y + P14K) | |
| SEQ ID NO: 86 | Nucleotide sequence of mVip3Aa | |
| | (A12S + P14V) | |
| SEQ ID NO: 87 | Nucleotide sequence of mVip3Aa | |
| | (A12H + P14Q) | |
| SEQ ID NO: 88 | Nucleotide sequence of mVip3Aa | |
| | (A12H + P14D) | |
| SEQ ID NO: 89 | Nucleotide sequence of mVip3Aa | |
| | (A12V + P14C) | |
| SEQ ID NO: 90 | Nucleotide sequence of mVip3Aa | |
| | (A12P + P14G) | |
| SEQ ID NO: 91 | Nucleotide sequence of mVip3Aa | |
| | (A12W + P14T) | |
| SEQ ID NO: 92 | Nucleotide sequence of mVip3Aa | |
| | (A12G + P14D) | |
| SEQ ID NO: 93 | Nucleotide sequence of mVip3Aa | |
| | (A12F + P14I) | |
| SEQ ID NO: 94 | Nucleotide sequence of mVip3Aa | |
| | (A12G + P14A) | |
| SEQ ID NO: 95 | Nucleotide sequence of mVip3Aa | |
| | (A12F + P14H) | |
| SEQ ID NO: 96 | Nucleotide sequence of mVip3Aa | |
| | (A12N + P14Q) | |
| SEQ ID NO: 97 | Nucleotide sequence of mVip3Aa | |
| | (A12Q + P14H) | |
| SEQ ID NO: 98 | Nucleotide sequence of mVip3Aa | |
| | (A12P + P14M) | |
| SEQ ID NO: 99 | Nucleotide sequence of mVip3Aa | |
| | (A12D + P14F) | |
| SEQ ID NO: 100 | Nucleotide sequence of mVip3Aa | |
| | (A12H + P14K) | |
| SEQ ID NO: 101 | Nucleotide sequence of mVip3Aa | |
| | (A12F + P14M) | |
| SEQ ID NO: 102 | Nucleotide sequence of mVip3Aa | |
| | (A12E) | |
| SEQ ID NO: 103 | Nucleotide sequence of mVip3Aa | |
| | (A12G + P14I) | |
| SEQ ID NO: 104 | Nucleotide sequence of mVip3Aa | |
| | (A12D + P14G) | |
| SEQ ID NO: 105 | Nucleotide sequence of mVip3Aa | |
| | (A12Q + P14V) | |
| SEQ ID NO: 106 | Nucleotide sequence of mVip3Aa | |
| | (A12S + P14G) | |
| SEQ ID NO: 107 | Nucleotide sequence of mVip3Aa | |
| | (A12F + P14G) | |
| SEQ ID NO: 108 | Nucleotide sequence of mVip3Aa | |
| | (A12G + P14S) | |
| SEQ ID NO: 109 | Nucleotide sequence of mVip3Aa | |
| | (A12R + P14M) | |
| SEQ ID NO: 110 | Nucleotide sequence of mVip3Aa | |
| | (A12H) | |
| SEQ ID NO: 111 | Nucleotide sequence of mVip3Aa | |
| | (A12T) | |
| SEQ ID NO: 112 | Nucleotide sequence of mVip3Aa | |
| | (P14C) | |
| SEQ ID NO: 113 | Nucleotide sequence of mVip3Aa | |
| | (P14V) | |
| SEQ ID NO: 114 | Nucleotide sequence of mVip3Aa | |
| | (P14A) | |
| SEQ ID NO: 115 | Nucleotide sequence of mVip3Aa | |
| | (P14D) | |
| SEQ ID NO: 116 | Nucleotide sequence of mVip3Aa | |
| | (P14E) | |
| SEQ ID NO: 117 | Nucleotide sequence of mVip3Aa | |
| | (P14F) | |
| SEQ ID NO: 118 | Nucleotide sequence of mVip3Aa | |
| | (P14H) | |
| SEQ ID NO: 119 | Nucleotide sequence of mVip3Aa | |
| | (P14I) | |
| SEQ ID NO: 120 | Nucleotide sequence of mVip3Aa | |
| | (P14K) | |

### DETAILED EMBODIMENTS OF THE INVENTION

The following examples are put forth so as to provide those skilled in the art with a complete disclosure and description of how to prepare and use the present invention, and are not intended to limit the scope of what the inventors regard as their invention, nor are they intended to represent or imply that the experiments below are all of or the only experiments performed. It will be appreciated by those skilled in the art that numerous variations and/or modifications may be made to the invention as shown in the specific aspects without departing from the spirit or scope of the invention as broadly described. Therefore, the present specification is considered in every respect as illustrative rather than restrictive.

### Example 1

### Construction of Maize Transgenic Vectors

According to the Vip3 Aa sequence information listed on the Bt gene nomenclature website (http://www.lifesci.sussex.ac.uk/home/Neil_Crickmore/Bt/vip.html), the Vip3Aa protein sequence (GenBank: ABG20429.1, see SEQ ID NO: 4 for its amino acid sequence) that performed well in transgenic maize mir162 was selected. After protein structure prediction, the 12th position of its amino acid sequence was mutated from Ala into Gly, and the 14th position was mutated from Pro into Gln. The protein was named Vip3Aa-K1 whose amino acid sequence is shown in SEQ ID NO: 1. Maize-codon optimization was performed on the amino acid sequence, and the corresponding coding nucleotide sequence is shown in SEQ ID NO: 2 which comprises 2,367 nucleotides and encodes 789 amino acids. The nucleotide sequence was sent to GenScript (Nanjing) Co., Ltd. for synthesis.

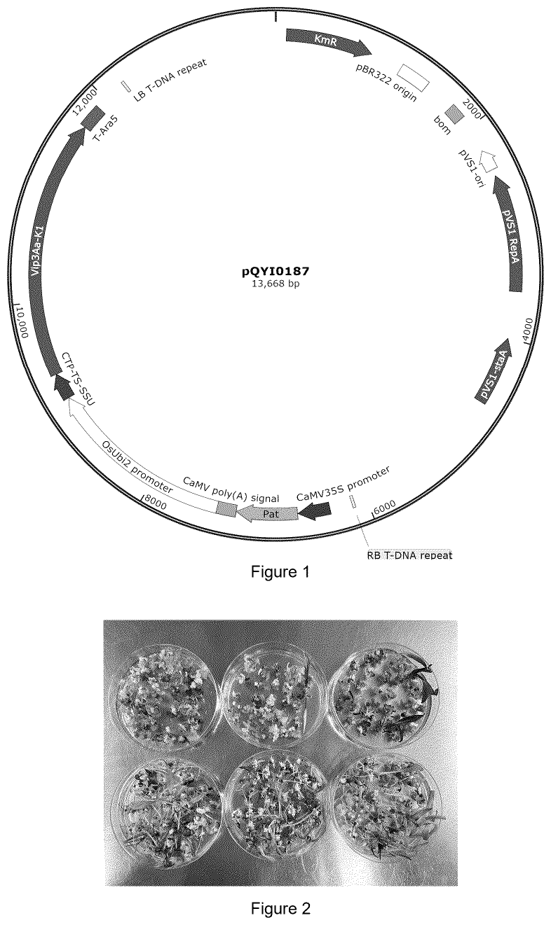

When the Vip3Aa-K1 gene sequence was artificially synthesized, the chloroplast localized peptide CTP-TS-SSU was simultaneously synthesized in the upstream of ATG, of which the nucleotide sequence is shown in SEQ ID NO: 3. The artificially synthesized CTP-TS-SSU-Vip3Aa-K1 gene fragment was constructed to the downstream of the rice Ubiquitin2 promoter and the upstream of the T-Ara5 terminator to obtain the Vip3Aa-K1 gene expression cassette which is initiated by the OsUbi2 promoter. The Vip3Aa-K1 gene expression cassette was then inserted into a vector containing the pat gene by homologous recombination-based seamless cloning method to obtain an expression cassette containing insect-resistant gene Vip3Aa-K1 and glufosinate-ammonium-resistant gene pat, and then the two expression cassettes were connected between LB and RB of the pCAMBIA1300 backbone using homologous recombination method to construct the vector pQYI0187 (FIG. 1).

The vector pQYI0186 was constructed according to the above-mentioned method, wherein the difference between pQYI0186 and pQYI0187 was that the Vip3Aa-K1 was replaced by the MIR162 Vip3 Aa.

Ten representative Vip3 family protein sequences (SEQ ID NO: 12, 14, 16, 18, 20, 22, 24, 26, 28, 30) were selected. The amino acid at position 2 in the amino acid sequence SEQ ID NO: 12 (corresponding to the amino acid at position 14 in the amino acid sequence set forth in SEQ ID NO: 4) was mutated from Pro into Gln. In the other 9 sequences, the amino acids at position 12 were mutated from Ala into Gly and the amino acids at position 14 were mutated from Pro into Gln. The amino acid sequences after mutations are respectively shown in SEQ ID NO: 11, 13, 15, 17, 19, 21, 23, 25, 27, 29. The above proteins and mutant proteins were respectively constructed into vectors pQYI0011-pQYI0030 according to the construction method of vector pQYI0187.

Furthermore, site-directed saturation mutation(s) of amino acid(s) at position 12 and/or at position 14 in the sequence of SEQ ID NO: 4 were generated separately or simultaneously. In addition to the above-mentioned SEQ ID NO: 1, a total of 398 new amino acid sequences were produced by the method of mutagenesis. According to the construction method of vector pQYI0186, the above 398 amino acid sequences were subject to maize-codon optimization to respectively construct vectors with double point mutations and with single point mutation for transformation of maize immature embryo calli.

### Example 2

### Comparison of Cytotoxicity of Transgenic Plants

The transgenic vectors pQYI0187 and pQYI0186 were transformed into maize calli by Agrobacterium transformation method, and the transformants QYI187 and QYI186 were obtained after screening and cultivation. During the process of genetic transformation, emergence of intermediate materials of QYI187 and QY1186 maize transformants was compared.

TABLE 1
Positive transformed seedlings of QYI187 and QYI186

| | Transformed | |
|---|---|---|
| Transformant with | immature embryo | Positive transformed |
| insect-resistant gene | (number) | seedling (strain) |
| QYI187 | 1000 | 630 |
| QYI186 | 1000 | 60 |

The results showed that the growth of the calli of QYI186 transformants was severely inhibited and harmed, and there were only 60 positive transformed seedlings among the 1000 transformed immature embryos; while the calli of QYI187 transformants grew well, and there were 630 positive transformed seedlings among the 1000 transformed immature embryos, as shown in FIG. 2 and Table 1. It indicated that the QYI187 (Vip3Aa-K1) transgenic materials had significant reduced plant cytotoxicity to recipient plants.

Similarly, the transgenic vectors pQYI0011-pQYI0030 were transformed into maize calli using Agrobacterium transformation method to obtain transformants QYI11-QYI30 after screening and cultivation. During the process of genetic transformation, emergence of intermediate materials of QYI11-QY130 maize transformants was compared. The results showed that the growth of the calli of unmutated QYI12 and QYI14 transformants was severely inhibited and harmed, and there were very few positive transformed seedlings among the 1,000 transformed immature embryos, 58 and 75 respectively; while the calli of QYI11 and QYI13 transformants comprising mutant proteins grew well, and there were respectively 614 and 571 positive transformed seedlings among the 1,000 transformed immature embryos. It indicated that QYI11 (double mutant of Vip3 Aa truncated protein) and QYI13 (double mutant of Vip3Aa19) transgenic materials comprising mutant proteins had significantly reduced plant cytotoxicity to recipient plants. Similarly, other transgenic materials (QYI15, QYI17, QYI19, QYI21, QYI23, QYI25, QYI27 and QYI29) with mutated Vip3 family proteins also exhibited significantly reduced plant cytotoxicity.

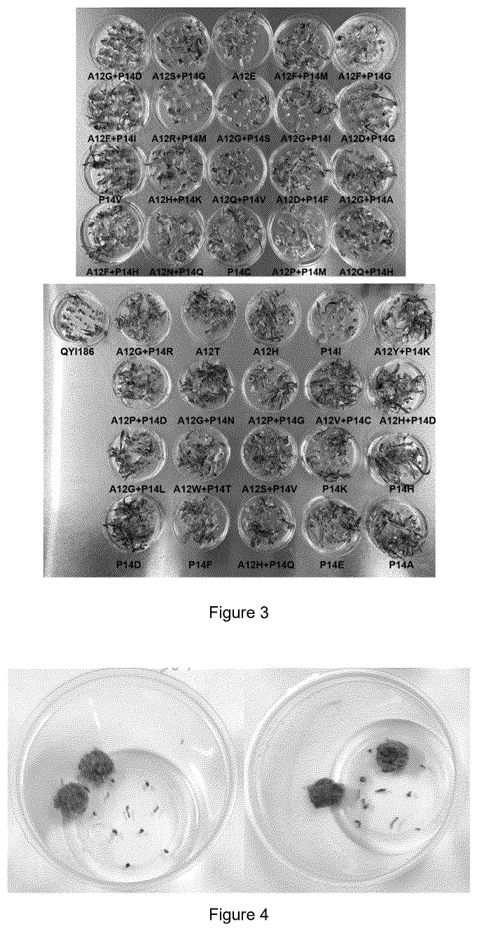

Furthermore, the intermediate materials of the transformants of the vectors with double point mutations and single point mutation constructed in Example 1 were also compared. The results showed that the numbers of positive seedlings of all exceeded that of QYI186 and the cytotoxicity of the mutants to plants was reduced. Wherein, when the amino acid(s) at position 12 and/or at position 14 in Vip3Aa (SEQ ID NO: 4) were mutated into 12P/14D, 12G/14N, 12G/14L, 12G/14R, 12Y/14K, 12S/14V, 12H/14Q, 12H/14D, 12V/14C, 12P/14G, 12W/14T, 12G/14D, 12F/14I, 12G/14A, 14V, 12F/14H, 14C, 12N/14Q, 12Q/14H, 12P/14M, 12D/14F, 12H/14K, 12F/14M, 12E, 12G/14I, 12D/14G, 12Q/14V, 12S/14G, 12F/14G, 12G/14S, 12R/14M, 12H, 12T, 14A, 14D, 14E, 14F, 14H, 14I, 14K, a large number of positive seedlings (338-626 positive transformed seedlings/1000 transformed immature embryos) were obtained and the intermediate materials of transformants grew normally on the screening culture dish, that was, the cytotoxicity to recipient plants was significantly reduced. The comparison of representative phytotoxicity is shown in FIG. 3.

### Example 3

### Assay of Protein Content in Leaves and Insecticidal Activity of Transgenic Maizes

According to protein expression level assay, at the V7-V8 stage of T2-generation transgenic maizes, the average expression level of Vip3 Aa-K1 in leaves of the transgenic maize QY1187 reached 130 μg/g (leaf fresh weight), and the average expression level of MIR162 Vip3 Aa in leaves of the transgenic maize QY1186 was 11 μg/g (leaf fresh weight). Moreover, it was found after experimentations that, the expression levels of the corresponding mutant proteins in other transgenic maizes were also distinctly higher than that in the corresponding transgenic maizes containing original proteins.

Leaves on the upper part of T2 generation QYI186 and QYI187 test maize plants which were at the V7-V8 stage and in the same growth state were cut, and respectively placed into sealed bags marked with corresponding names. The leaves were taken back to the lab and cut into a size of 2 cm², then put into different mortars respectively and ground with liquid nitrogen. The frozen leaf powders were diluted to 4× and 50× multiples; when the temperature of the feed to be prepared was reduced to 45° C., frozen leaf powders were added in proportion and stirred evenly; after cooled down completely, the feed was made into round cakes of uniform shape and weight using a fixed mould and put into a bioassay device each of which was inoculated with a 2^{nd} instar Spodoptera frugiperda larva. Ten replicates were set. The experiment was carried out in an insectary under the conditions that temperature of 27±1° C., RH of 75% and L: D=16 h: 8 h. The death of larvae in each device was investigated after 7 days.

The 2^{nd} instar Spodoptera frugiperda larvae in the 10 replicates were collected into an experimental device, and the results are as shown in FIG. 4. The insecticidal protein concentrations of QY1187 diluted 50 times and QY1186 diluted 4 times were 2.6 μg/g (diet fresh weight) and 2.75 μg/g (diet fresh weight) respectively, where the concentrations of Vip3Aa-K1 and Vip3 Aa contained were comparable. The death rates of 2^{nd} instar Spodoptera frugiperda larvae were both 100%.

In addition, the median lethal concentration (LC50) of each original protein and each mutant protein to Spodoptera frugiperda was determined by the diet surface method. The newly-made artificial diet was poured into a beaker, soaked in hot water, and distributed into a 24-well cell culture plate using a manual pipette repeater, in which each well was filled with 1 mL of diet and the diameter of the well was 1.6 cm. After the diet solidified, the surface area formed was 2 cm². The mutant protein to be tested was subject to gradient dilution with Na₂CO₃/NaHCO₃ buffer (pH=10) to 4 concentrations. The dilutions of above concentrations were distributed into each well with 50 μl using a manual pipette repeater and shaken well, allowing the protein to completely cover the diet surface. The 24-well cell culture plate loaded with samples was placed in an ultra-clean workbench to blow-dry. After the protein had permeated into the diet surface, 2^{nd} instar Spodoptera frugiperda larvae were inoculated. Each well was inoculated with 1 insect. The plate was covered with a lid, tied tightly, and placed under conditions of a temperature of 25-27° C., a relative humidity of 65-70%, and illumination of L/D=16h/8h. With the plate added with 50 μl of buffer solution as a control, the procedure was repeated twice. After 7 days, the death of test insects in each group was observed and the death rates of insects were counted to work out the corresponding median lethal concentrations (LC50). The results showed that LC50 values of the mutant proteins of the present invention were not significantly different from those of their corresponding original Vip3 proteins, and the insecticidal effects on Spodoptera frugiperda were sustained or even improved. The representative data are shown in Table 2.

TABLE 2
Median lethal concentration (LC50) data of some
Vip3 mutant proteins to Spodoptera frugiperda

| | LC50 (95% confidence interval) |
|---|---|
| Mutation Site | μg/cm² |
| MIR162 Vip3Aa (QYI186) | 0.34 (0.31-0.39) |
| Vip3Aa-K1 (QYI187) | 0.38 (0.33-0.47) |
| Vip3Aa19 (QYI14) | 1.11 (0.94-1.34) |
| Double mutant of Vip3Aa19 (QYI13) | 1.20 (0.82-1.98) |
| Vip3Aa truncated protein (QYI12) | 0.37 (0.27-0.46) |
| Double mutant of Vip3Aa truncated | 0.43 (0.34-0.52) |
| protein (QYI11) | |
| mVip3Aa (A12P + P14D) | 0.39 (0.32-0.42) |
| mVip3Aa (A12G + P14N) | 0.36 (0.31-0.40) |
| mVip3Aa (A12G + P14L) | 0.37 (0.34-0.42) |
| mVip3Aa (A12G + P14R) | 0.25 (0.21-0.38) |
| mVip3Aa (A12Y + P14K) | 0.29 (0.21-0.37) |
| mVip3Aa (A12S + P14V) | 0.33 (0.30-0.38) |
| mVip3Aa (A12H + P14Q) | 0.35 (0.28-0.41) |
| mVip3Aa (A12H + P14D) | 0.27 (0.12-0.45) |
| mVip3Aa (A12V + P14C) | 0.32 (0.21-0.41) |
| mVip3Aa (A12P + P14G) | 0.39 (0.29-0.56) |
| mVip3Aa (A12W + P14T) | 0.45 (0.38-0.58) |
| mVip3Aa (A12G + P14D) | 0.32 (0.26-0.40) |
| mVip3Aa (A12F + P14I) | 0.39 (0.31-0.51) |
| mVip3Aa (A12G + P14A) | 0.31 (0.24-0.43) |
| mVip3Aa (A12F + P14H) | 0.31 (0.20-0.61) |
| mVip3Aa (A12N + P14Q) | 0.42 (0.28-0.71) |
| mVip3Aa (A12Q + P14H) | 0.38 (0.23-0.65) |
| mVip3Aa (A12P + P14M) | 0.28 (0.17-0.44) |
| mVip3Aa (A12D + P14F) | 0.21 (0.17-0.38) |
| mVip3Aa (A12H + P14K) | 0.36 (0.28-0.68) |
| mVip3Aa (A12F + P14M) | 0.25 (0.16-0.44) |
| mVip3Aa (A12E) | 0.37 (0.14-0.47) |
| mVip3Aa (A12G + P14I) | 0.40 (0.28-0.92) |
| mVip3Aa (A12D + P14G) | 0.34 (0.27-0.45) |
| mVip3Aa (A12Q + P14V) | 0.32 (0.24-0.49) |
| mVip3Aa (A12S + P14G) | 0.27 (0.20-0.49) |
| mVip3Aa (A12F + P14G) | 0.37 (0.34-0.73) |
| mVip3Aa (A12G + P14S) | 0.29 (0.18-0.52) |
| mVip3Aa (A12R + P14M) | 0.39 (0.27-0.71) |
| mVip3Aa (A12H) | 0.42 (0.37-0.49) |
| mVip3Aa (A12T) | 0.38 (0.33-0.57) |
| mVip3Aa (P14C) | 0.36 (0.31-0.59) |
| mVip3Aa (P14V) | 0.37 (0.35-0.52) |
| mVip3Aa (P14A) | 0.28 (0.19-0.34) |
| mVip3Aa (P14D) | 0.19 (0.15-0.44) |
| mVip3Aa (P14E) | 0.32 (0.24-0.39) |
| mVip3Aa (P14F) | 0.26 (0.18-0.37) |
| mVip3Aa (P14H) | 0.29 (0.21-0.40) |
| mVip3Aa (P14I) | 0.32 (0.23-0.38) |
| mVip3Aa (P14K) | 0.35 (0.26-0.42) |

### Example 4

### Assay of Insect-Resistant Effects of Transgenic Soybean Leaves

Based on the same method of Example 1, soybean transgenic vectors comprising Vip3Aa-K1 was constructed and transformed into soybean recipients to obtain transgenic soybeans. Leaves on the upper part of test soybean plants which were in the same growth state were cut and placed into sealed bags marked with corresponding names. The leaves were taken back to the lab and cut into a size of 2 cm², then put into a bioassay device each of which was inoculated with three 2^{nd} instar Helicoverpa armigera larvae. Ten replicates were set. The experiment was carried out in an insectary under the conditions that temperature of 27±1° C., RH of 75% and L: D=16 h: 8 h. The death of larvae and leaf feeding in each device were investigated after 6 days.

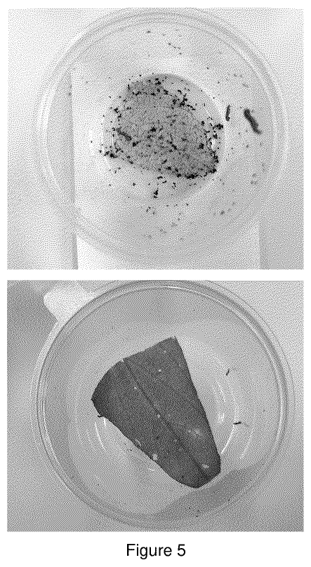

The experimental results showed that the non-transgenic soybean leaves were severely eaten by Helicoverpa armigera larvae, and the larvae grew well; while the leaves of transgenic soybeans transformed with Vip3Aa-K1 were barely eaten, and the larvae all died after eating, which indicated that the transgenic soybean plants transformed with Vip3Aa-K1 had better resistance phenotype against Helicoverpa armigera larvae as shown in FIG. 5.

Meanwhile, it has been found after extensive experimentations that, the transgenic plants (including but not limited to maize, cotton, soybean, etc.) comprising the Vip3 Aa-K1 protein of the present invention exhibited similar insect-resistant effects in experiments against other lepidopteran plant pests in the Genus Spodoptera, Striacosta, Agrotis, Dichocrocis, Mythimna and Elasmopalpus, and the other mutant Vip3 family proteins also had excellent insecticidal effects on various pests and low toxicity to plant cells.

Finally, it should be noted that the above embodiments are only to illustrate the technical solutions of the present invention and not to set limitations. Although the present invention has been described in detail with reference to preferred embodiments, it should be understood by one of ordinary skill in the art that the technical solutions of the present invention can be modified or equivalently replaced without departing from the spirit and scope of the technical solutions of the present invention.

1-17. (canceled)

18. A mutant insecticidal protein Vip3 comprising an amino acid sequence having the following mutation(s) compared with the amino acid sequence of any Vip3 family protein: the amino acid(s) corresponding to the amino acid at position 12 in the amino acid sequence set forth in SEQ ID NO: 4 being mutated into any other amino acid and/or to the amino acid at position 14 being mutated into any other amino acid.

19. The mutant insecticidal protein Vip3 according to claim 18, wherein it is characterized in that, it comprises an amino acid sequence having the following mutation(s) compared with the amino acid sequence of any Vip3 family protein: the amino acid(s) corresponding to the amino acid at position 12 in the amino acid sequence set forth in SEQ ID NO: 4 being mutated from alanine into glycine, valine, leucine, isoleucine, methionine, proline, tryptophan, serine, tyrosine, cysteine, phenylalanine, asparagine, glutamine, threonine, aspartic acid, glutamic acid, lysine, arginine or histidine; and/or to the amino acid at position 14 being mutated from proline into alanine, glycine, valine, leucine, isoleucine, methionine, tryptophan, serine, tyrosine, cysteine, phenylalanine, asparagine, glutamine, threonine, aspartic acid, glutamic acid, lysine, arginine or histidine.

20. The mutant insecticidal protein Vip3 according to claim 18, wherein it is characterized in that, the amino acid sequence of the Vip3 family protein is set forth in SEQ ID NO: 4, SEQ ID NO: 12, SEQ ID NO: 14, SEQ ID NO: 16, SEQ ID NO: 18, SEQ ID NO:
20, SEQ ID NO: 22, SEQ ID NO: 24, SEQ ID NO: 26, SEQ ID NO: 28 or SEQ ID NO: 30.

21. The mutant insecticidal protein Vip3 according to claim 18, comprising an amino acid sequence having the following mutation(s) compared with the amino acid sequence of any Vip3 family protein:
the amino acid(s) corresponding to the amino acid at position 12 in the amino acid sequence set forth in SEQ ID NO: 4 being mutated from alanine into glycine and/or to the amino acid at position 14 being mutated from proline into glutamine;
the amino acid(s) corresponding to the amino acid at position 12 in the amino acid sequence set forth in SEQ ID NO: 4 being mutated from alanine into proline and/or to the amino acid at position 14 being mutated from proline into aspartic acid;
the amino acid(s) corresponding to the amino acid at position 12 in the amino acid sequence set forth in SEQ ID NO: 4 being mutated from alanine into glycine and/or to the amino acid at position 14 being mutated from proline into asparagine;
the amino acid(s) corresponding to the amino acid at position 12 in the amino acid sequence set forth in SEQ ID NO: 4 being mutated from alanine into glycine and/or to the amino acid at position 14 being mutated from proline into leucine;
the amino acid(s) corresponding to the amino acid at position 12 in the amino acid sequence set forth in SEQ ID NO: 4 being mutated from alanine into glycine and/or to the amino acid at position 14 being mutated from proline into arginine;
the amino acid(s) corresponding to the amino acid at position 12 in the amino acid sequence set forth in SEQ ID NO: 4 being mutated from alanine into tyrosine and/or to the amino acid at position 14 being mutated from proline into lysine;
the amino acid(s) corresponding to the amino acid at position 12 in the amino acid sequence set forth in SEQ ID NO: 4 being mutated from alanine into serine and/or to the amino acid at position 14 being mutated from proline into valine;
the amino acid(s) corresponding to the amino acid at position 12 in the amino acid sequence set forth in SEQ ID NO: 4 being mutated from alanine into histidine and/or to the amino acid at position 14 being mutated from proline into glutamine;
the amino acid(s) corresponding to the amino acid at position 12 in the amino acid sequence set forth in SEQ ID NO: 4 being mutated from alanine into histidine and/or to the amino acid at position 14 being mutated from proline into aspartic acid;
the amino acid(s) corresponding to the amino acid at position 12 in the amino acid sequence set forth in SEQ ID NO: 4 being mutated from alanine into valine and/or to the amino acid at position 14 being mutated from proline into cysteine;
the amino acid(s) corresponding to the amino acid at position 12 in the amino acid sequence set forth in SEQ ID NO: 4 being mutated from alanine into proline and/or to the amino acid at position 14 being mutated from proline into glycine;
the amino acid(s) corresponding to the amino acid at position 12 in the amino acid sequence set forth in SEQ ID NO: 4 being mutated from alanine into tryptophan and/or to the amino acid at position 14 being mutated from proline into threonine;
the amino acid(s) corresponding to the amino acid at position 12 in the amino acid sequence set forth in SEQ ID NO: 4 being mutated from alanine into glycine and/or to the amino acid at position 14 being mutated from proline into aspartic acid;
the amino acid(s) corresponding to the amino acid at position 12 in the amino acid sequence set forth in SEQ ID NO: 4 being mutated from alanine into phenylalanine and/or to the amino acid at position 14 being mutated from proline into isoleucine;
the amino acid(s) corresponding to the amino acid at position 12 in the amino acid sequence set forth in SEQ ID NO: 4 being mutated from alanine into glycine and/or to the amino acid at position 14 being mutated from proline into alanine;
the amino acid(s) corresponding to the amino acid at position 12 in the amino acid sequence set forth in SEQ ID NO: 4 being mutated from alanine into phenylalanine and/or to the amino acid at position 14 being mutated from proline into histidine;
the amino acid(s) corresponding to the amino acid at position 12 in the amino acid sequence set forth in SEQ ID NO: 4 being mutated from alanine into asparagine and/or to the amino acid at position 14 being mutated from proline into glutamine;
the amino acid(s) corresponding to the amino acid at position 12 in the amino acid sequence set forth in SEQ ID NO: 4 being mutated from alanine into glutamine and/or to the amino acid at position 14 being mutated from proline into histidine;
the amino acid(s) corresponding to the amino acid at position 12 in the amino acid sequence set forth in SEQ ID NO: 4 being mutated from alanine into proline and/or to the amino acid at position 14 being mutated from proline into methionine;
the amino acid(s) corresponding to the amino acid at position 12 in the amino acid sequence set forth in SEQ ID NO: 4 being mutated from alanine into aspartic acid and/or to the amino acid at position 14 being mutated from proline into phenylalanine;
the amino acid(s) corresponding to the amino acid at position 12 in the amino acid sequence set forth in SEQ ID NO: 4 being mutated from alanine into histidine and/or to the amino acid at position 14 being mutated from proline into lysine;
the amino acid(s) corresponding to the amino acid at position 12 in the amino acid sequence set forth in SEQ ID NO: 4 being mutated from alanine into phenylalanine and/or to the amino acid at position 14 being mutated from proline into methionine;
the amino acid corresponding to the amino acid at position 12 in the amino acid sequence set forth in SEQ ID NO: 4 being mutated from alanine into glutamic acid;
the amino acid(s) corresponding to the amino acid at position 12 in the amino acid sequence set forth in SEQ ID NO: 4 being mutated from alanine into glycine and/or to the amino acid at position 14 being mutated from proline into isoleucine;
the amino acid(s) corresponding to the amino acid at position 12 in the amino acid sequence set forth in SEQ ID NO: 4 being mutated from alanine into aspartic acid and/or to the amino acid at position 14 being mutated from proline into glycine;
the amino acid(s) corresponding to the amino acid at position 12 in the amino acid sequence set forth in SEQ ID NO: 4 being mutated from alanine into glutamine and/or to the amino acid at position 14 being mutated from proline into valine;
the amino acid(s) corresponding to the amino acid at position 12 in the amino acid sequence set forth in SEQ ID NO: 4 being mutated from alanine into serine and/or to the amino acid at position 14 being mutated from proline into glycine;
the amino acid(s) corresponding to the amino acid at position 12 in the amino acid sequence set forth in SEQ ID NO: 4 being mutated from alanine into phenylalanine and/or to the amino acid at position 14 being mutated from proline into glycine;
the amino acid(s) corresponding to the amino acid at position 12 in the amino acid sequence set forth in SEQ ID NO: 4 being mutated from alanine into glycine and/or to the amino acid at position 14 being mutated from proline into serine;
the amino acid(s) corresponding to the amino acid at position 12 in the amino acid sequence set forth in SEQ ID NO: 4 being mutated from alanine into arginine and/or to the amino acid at position 14 being mutated from proline into methionine;
the amino acid corresponding to the amino acid at position 12 in the amino acid sequence set forth in SEQ ID NO: 4 being mutated from alanine into histidine;
the amino acid corresponding to the amino acid at position 12 in the amino acid sequence set forth in SEQ ID NO: 4 being mutated from alanine into threonine;
the amino acid corresponding to the amino acid at position 14 in the amino acid sequence set forth in SEQ ID NO: 4 being mutated from proline into cysteine;
the amino acid corresponding to the amino acid at position 14 in the amino acid sequence set forth in SEQ ID NO: 4 being mutated from proline into valine;
the amino acid corresponding to the amino acid at position 14 in the amino acid sequence set forth in SEQ ID NO: 4 being mutated from proline into alanine;
the amino acid corresponding to the amino acid at position 14 in the amino acid sequence set forth in SEQ ID NO: 4 being mutated from proline into aspartic acid;
the amino acid corresponding to the amino acid at position 14 in the amino acid sequence set forth in SEQ ID NO: 4 being mutated from proline into glutamic acid;
the amino acid corresponding to the amino acid at position 14 in the amino acid sequence set forth in SEQ ID NO: 4 being mutated from proline into phenylalanine;
the amino acid corresponding to the amino acid at position 14 in the amino acid sequence set forth in SEQ ID NO: 4 being mutated from proline into histidine;
the amino acid corresponding to the amino acid at position 14 in the amino acid sequence set forth in SEQ ID NO: 4 being mutated from proline into isoleucine: or, the amino acid corresponding to the amino acid at position 14 in the amino acid sequence set forth in SEQ ID NO: 4 being mutated from proline into lysine.

22. The mutant insecticidal protein Vip3 according to claim 18, the amino acid sequence of which is as set forth in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO:
25, SEQ ID NO: 27, SEQ ID NO: 29 or SEQ ID NO: 41-80.

23. An isolated polynucleotide comprising a nucleic acid sequence encoding the mutant insecticidal protein Vip3 according to claim 18, or a complementary sequence thereof.

24. The polynucleotide according to claim 23, wherein it is characterized in that the polynucleotide is DNA, RNA, or a hybrid thereof.

25. The polynucleotide according to claim 23, wherein it is characterized in that the polynucleotide is single-stranded or double-stranded.

26. The polynucleotide according to claim 23, wherein it is characterized in that it has a nucleic acid sequence selected from:
(1) a nucleic acid sequence encoding the amino acid sequence as shown in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29 or SEQ ID NO: 41-80, or a complementary sequence thereof;
(2) the nucleic acid sequence as shown in any one of SEQ ID NO: 2, SEQ ID NO: 31-40, SEQ ID NO: 81-120, or a complementary sequence thereof;
(3) a nucleic acid sequence that hybridizes to the sequence shown in (1) or (2) under stringent conditions; and/or
(4) a nucleic acid sequence encoding the same amino acid sequence as the sequence shown in (1) or (2) due to degeneracy of the genetic code, or a complementary sequence thereof.

27. The polynucleotide according to claim 26, wherein the nucleic acid sequence is optimized for expression in plant cells.

28. An expression vector comprising the polynucleotide according to claim 23 and an expression regulatory element operably linked thereto.

29. An expression vector comprising gene tandem expression cassettes that express the mutant insecticidal protein Vip3 according to claim 18 and Pat.

30. The expression vector according to claim 29, the nucleotide sequence of the genetically mutant insecticidal protein Vip3 is shown in any one of SEQ ID NO: 2, SEQ ID NO: 31-40, SEQ ID NO: 81-120 and the nucleotide sequence of the pat gene is SEQ ID NO: 6.

31. The expression vector according to claim 29, wherein the gene tandem expression cassettes also comprise:
a CaMV 35S promoter which initiates the expression of the pat and of which the nucleotide sequence is as shown in SEQ ID NO: 5, and a CaMV poly (A) signal termination sequence which terminates the expression of the gene and of which the nucleotide sequence is as shown in SEQ ID NO: 7;
an OsUbi2 promoter which initiates the expression of the mutant insecticidal protein Vip3 and of which the nucleotide sequence is as shown in SEQ ID NO: 8, a chloroplast leading peptide CTP-TS-SSU whose nucleotide sequence is as shown in SEQ ID NO: 3, and a T-Ara5 terminator which terminates the expression of the gene and of which the nucleotide sequence is as shown in SEQ ID NO: 9;
preferably, the nucleotide sequence of the expression vector is as shown in SEQ ID NO: 10.

32. A host cell comprising the polynucleotide according to claim 6 or an expression vector comprising the polynucleotide.

33. The host cell according to claim 32, the host cell is a plant cell.

34. A method of cultivating a transgenic plant having or with enhanced insect resistance and a plant produced by the method, wherein it includes regenerating the plant cell according to claim 33 into a plant.

35. A method of managing insect resistance or controlling an insect, wherein it is characterized in that it comprises contacting the insect with at least the plant according to claim 34, wherein the insect contacts at least the mutant insecticidal protein Vip3 by ingesting tissues of the plant, after which the insect is inhibited in growth and/or died, thereby achieving management of resistance against the insect or achieving control of the insect damaging the plant.

36. The method according to claim 35, wherein the plant is preferably maize, cotton or soybean, and the insect is preferably a lepidopteran.


SEQUENCE LISTING (120 sequences)

SEQ ID NO: 1 - protein, 789 aa, synthetic construct
MNKNNTKLSTRGLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 2 - DNA, 2367 bp, synthetic construct
  source 1..2367: mol_type = other DNA
  CDS 1..2367: protein_id = 1; transl_table = 1; translation = MNKNNTKLSTRGLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgcaaagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 3 - DNA, 240 bp, synthetic construct
  source 1..240: mol_type = other DNA
accatggcgcccaccgtgatgatggcctcgtcggccaccgccgtcgcttcgttccaggggctcaagtccaccgccagcctccccgtcgcccgccgctcctccagaagcctcggcaacgtcagcaacggcggaaggatccggtgcatgcaggtgtggccggcctacggcaacaagaagttcgagacgctgtcgtacctgccgccgctgtccaccggcgggcgcatccgctgcatgcaggcc

SEQ ID NO: 4 - protein, 789 aa, synthetic construct
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 5 - DNA, 293 bp, synthetic construct
  source 1..293: mol_type = other DNA
ccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagagg

SEQ ID NO: 6 - DNA, 555 bp, synthetic construct
  source 1..555: mol_type = other DNA
atgtcgcccgagcgcaggcccgtcgagatccgcccggccacagcagccgacatggctgcggtgtgcgacatcgtcaaccactacatcgaaacgtccaccgtgaacttccgcaccgagccgcagacgccgcaggagtggatcgacgaccttgagcgcctccaggaccgctacccctggctcgtcgcggaggtcgaaggcgtcgtggccggcatcgcctacgccgggccgtggaaggctcgcaacgcctacgactggaccgtcgagagcaccgtctacgtgtcccaccgccaccagcgcctgggcctcggctccacgctgtacacgcacctgctcaagtcgatggaggcgcaaggcttcaagagcgtggtcgccgtcatcggcctcccgaacgatccctccgtgaggctccacgaagccctgggctacaccgctcggggcacgctgcgcgcagccggctacaagcacggtggctggcacgacgtcgggttctggcagagggacttcgagctgcccgcgcctccgcgcccggttcgccccgtgacccagatttgatag

SEQ ID NO: 7 - DNA, 175 bp, synthetic construct
  source 1..175: mol_type = other DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 8 - DNA, 1710 bp, synthetic construct
  source 1..1710: mol_type = other DNA
attcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaacaatatatagagaagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatccgtgatgtgcctgttactatctttttgaatacatgtatgttctgttggaatatgtttgctgtttgatccgttgttgtgtccttaatcttgtgctagttcttaccctatctgtttggtgattatttcttgcag

SEQ ID NO: 9 - DNA, 213 bp, synthetic construct
  source 1..213: mol_type = other DNA
ctaggctactgtagctagctgtgcatgtatgtggtgtggttactaaaataattagtgttttccttttgtttggaagcatatgtgtggtgaataaatgatgaactccgatgttcctctctataaatcttgatgattcgctagctatccgtacgtcgttgttctttgatttgatgatgagattgaaaaatggaatgtcatgctaaggagggtgcc

SEQ ID NO: 10 - DNA, 13668 bp, synthetic construct
  source 1..13668: mol_type = other DNA
gttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgaaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccccatcaagctagcttctgcaggtcctgctcgagcggccgctctagaactagtggatcccccccttaattaagggggctgcaggaattcgatatcaagcttctgcacctaggcgccagttaatagtttgacgcgtgcaacgttgttgccaatgtcgacccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggaccatgtcgcccgagcgcaggcccgtcgagatccgcccggccacagcagccgacatggctgcggtgtgcgacatcgtcaaccactacatcgaaacgtccaccgtgaacttccgcaccgagccgcagacgccgcaggagtggatcgacgaccttgagcgcctccaggaccgctacccctggctcgtcgcggaggtcgaaggcgtcgtggccggcatcgcctacgccgggccgtggaaggctcgcaacgcctacgactggaccgtcgagagcaccgtctacgtgtcccaccgccaccagcgcctgggcctcggctccacgctgtacacgcacctgctcaagtcgatggaggcgcaaggcttcaagagcgtggtcgccgtcatcggcctcccgaacgatccctccgtgaggctccacgaagccctgggctacaccgctcggggcacgctgcgcgcagccggctacaagcacggtggctggcacgacgtcgggttctggcagagggacttcgagctgcccgcgcctccgcgcccggttcgccccgtgacccagatttgatagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaacaatatatagagaagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatccgtgatgtgcctgttactatctttttgaatacatgtatgttctgttggaatatgtttgctgtttgatccgttgttgtgtccttaatcttgtgctagttcttaccctatctgtttggtgattatttcttgcagaccatggcgcccaccgtgatgatggcctcgtcggccaccgccgtcgcttcgttccaggggctcaagtccaccgccagcctccccgtcgcccgccgctcctccagaagcctcggcaacgtcagcaacggcggaaggatccggtgcatgcaggtgtggccggcctacggcaacaagaagttcgagacgctgtcgtacctgccgccgctgtccaccggcgggcgcatccgctgcatgcaggccatgaacaagaacaacacgaagctctccacccgcggcctgcaaagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaagtagtgactaggctactgtagctagctgtgcatgtatgtggtgtggttactaaaataattagtgttttccttttgtttggaagcatatgtgtggtgaataaatgatgaactccgatgttcctctctataaatcttgatgattcgctagctatccgtacgtcgttgttctttgatttgatgatgagattgaaaaatggaatgtcatgctaaggagggtgccggtaccaagctttggcgcgccaaatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttcctagcttggccggccatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacctggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattaccccttcggtcctccgatcgccgcggtttcaaaatcggctccgtcgatactatgttatacgccaactttgaaaacaactttgaaaaagctgttttctggtatttaag

SEQ ID NO: 11 - protein, 778 aa, synthetic construct
MLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 12 - protein, 778 aa, synthetic construct
MLPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 13 - protein, 789 aa, synthetic construct
MNKNNTKLSTRGLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 14 - protein, 789 aa, synthetic construct
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 15 - protein, 789 aa, synthetic construct
MNKNNTKLSTRGLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISEAFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWEDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKLGKDNFYIELSQGNNLNGGPIVKFSDVSIK

SEQ ID NO: 16 - protein, 789 aa, synthetic construct
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISEAFLTPINGFGLQADENSRLITLTCKSYLRELLLATDLSNKETKLIVPPSGFIKNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTITKRFTTGTDLKGVYLILKSQNGDEAWEDNFIILEISPSEKLLSPELINTNNWTSTGSTNISGNTLTLYQGGRGILKQNLQLDSFSTYRVYFSVSGDANVRIRNSREVLFEKRYMSGAKDVSEIFTTKLGKDNFYIELSQGNNLNGGPIVKFSDVSIK

SEQ ID NO: 17 - protein, 789 aa, synthetic construct
MNKNNTKLSTRGLQSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLAGIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 18 - protein, 789 aa, synthetic construct
MNKNNTKLSTRALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLAGIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDTDKLFCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 19 - protein, 789 aa, synthetic construct
MNMNNTKLNARGLQSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNFYIELSQGNNLYGGPLVKFNDVSIK

SEQ ID NO: 20 - protein, 789 aa, synthetic construct
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNFYIELSQGNNLYGGPLVKFNDVSIK

SEQ ID NO: 21 - protein, 789 aa, synthetic construct
MNMNNTKLNARGLQSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNPYIELSQGNNLYGGPLVKPNDVSIK

SEQ ID NO: 22 - protein, 789 aa, synthetic construct
MNMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEMSNDSITVLKVYEAKLKQNYQVDKDSLSEVIYGDMDKLLCPDQSEQIYYTNNIVFPNEYVITKIDFTKKMKTLRYEVTANSYDSSTGEIDLNKKKVESSEAEYRTLSAKDDGVYMPLGVISETFLTPINGFGLQADENSRLITLTCKSYLRELLLATDLSNKETKLIVPPSGFISNIVENGNLEGENLEPWIANNKNAYVDHTGGVNGTRALYVHKDGGFSQFIGDKLKPKTEYVIQYTVKGKPSIHLKNENTGYIHYEDTNNNLEDYQTITKRFTTGTDLKGVYLILKSQNGDEAWGDNFTILEISPSEKLLSPELINVNNWIRTGSTHISGNTLTLYQGGGGNLKQNLQLDSFSTYRVNFSVTGDANVRIRNSREVLFEKRYMSGAKDVSEIFTTKLGKDNPYIELSQGNNLYGGPLVKPNDVSIK

SEQ ID NO: 23 - protein, 788 aa, synthetic construct
MNKNNTKLSTRGLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSRGVLFERSNLMSSTSHISGTFKTESNNTGLYVELSRRSGGGGHISFENVSIK

SEQ ID NO: 24 - protein, 788 aa, synthetic construct
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDKFTILEIKPAEDLLSPELINPNSWITTPGASISGNKLFINLGTNGTFRQSLSLNSYSTYSISFTASGPFNVTVRNSRGVLFERSNLMSSTSHISGTFKTESNNTGLYVELSRRSGGGGHISFENVSIK

SEQ ID NO: 25 - protein, 786 aa, synthetic construct
MNMNNAKLNARGLQSFIDYFNGIYGFAIGIKDIMNMIFKTDTGSNLTLDEILKNQQLLNEISGKLDGVNGSLNDLIAQGNLNTELAKQILKVANEQNQVLNDVNNKLDTINSMLKIYLPKITSMLSDVMKQNYVLSLQIEYLSKQLQEISDKLDIINVNVLINSTLTEITPAYQRMKYVNEKFEELTFATETTLKVKKDSPPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKAKGSNEDTKMIVEAKPGYVLVGFEMSNDSITVLKAYQAKLKKDYQIDKDSLSEIIYSDTDKLLCPDQSEQIYYTKNIAFPNEYVITKIAFTKKMNSLRYEATANFYDSSTGDIDLNKTKVESSEAEYSMLKASDDEVYMPLGLISETFLNPINGFRLAVDENSRLVTLTCRSYLRETLLATDLNNKETKLIVPPNVFISNIVENGNIEMDTLEPWKANNENANVDYSGGVNGTRALYVHKDGEFSHFIGDKLKSKTEYLIRYIVKGKASIFLKDEKNENYIYEDTNNNLEDYQTITKRFTTGTDSTGVYLIFNSQNGDEAWGDNFIILEISPCEKLLSPELIKTDKWNSTGSTYISDDRLTLYRGGRGILKQNLQLDGFSTYRVNFSVDGDANVRIRNSREVLLEKRYLNRKGVSEMFTTKFDKDNFYVELSQGDNLGTIVHFYDFSIK

SEQ ID NO: 26 - protein, 786 aa, synthetic construct
MNMNNAKLNARALPSFIDYFNGIYGFAIGIKDIMNMIFKTDTGSNLTLDEILKNQQLLNEISGKLDGVNGSLNDLIAQGNLNTELAKQILKVANEQNQVLNDVNNKLDTINSMLKIYLPKITSMLSDVMKQNYVLSLQIEYLSKQLQEISDKLDIINVNVLINSTLTEITPAYQRMKYVNEKFEELTFATETTLKVKKDSPPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKAKGSNEDTKMIVEAKPGYVLVGFEMSNDSITVLKAYQAKLKKDYQIDKDSLSEIIYSDTDKLLCPDQSEQIYYTKNIAFPNEYVITKIAFTKKMNSLRYEATANFYDSSTGDIDLNKTKVESSEAEYSMLKASDDEVYMPLGLISETFLNPINGFRLAVDENSRLVTLTCRSYLRETLLATDLNNKETKLIVPPNVFISNIVENGNIEMDTLEPWKANNENANVDYSGGVNGTRALYVHKDGEFSHFIGDKLKSKTEYLIRYIVKGKASIFLKDEKNENYIYEDTNNNLEDYQTITKRFTTGTDSTGVYLIFNSQNGDEAWGDNFIILEISPCEKLLSPELIKTDKWNSTGSTYISDDRLTLYRGGRGILKQNLQLDGFSTYRVNFSVDGDANVRIRNSREVLLEKRYLNRKGVSEMFTTKFDKDNFYVELSQGDNLGTIVHFYDFSIK

SEQ ID NO: 27 - protein, 795 aa, synthetic construct
MNKNNTKLSTRGLQSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETNLKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYIKTKGSDEDAEVIIQAEPGHALVGFEMINDPSPALKVYQAKLKPNYQVDKQSLSETVYGDMDKILCPDKSQQMYYLHNITFPNEYVITEIIFTKKKNSLRYEVIANYYEFSSGDIDLNKKLVKSSEAEYSTLSVSNDAIYMPLGVISETFLTPIKGFGLTVDESSRLVTLTCKSYLREILLATDLSNKATKLIVPPNGFISNLVENGDIEADNIEPWKGNNKNAYVDHTGGVNGTKALYTQDDGEFSQFIGDKLKSKTEYIIQYTVKGNTSIYLKDKKNENVIYEDKNNNLEAFQTITKRFTTELDSSDVYLVFKCKNGYKAWGDNFLITEIRPKEVVSPELIKVENWIGMGGSNHVNPDSLLLFTGGRSILKQNLQLDSYSTYRVRFSLMVIGKAKVIIRNSSEVLFEKSYVNDSEGVLEGVSETFTTKSIQDNFYVELSNEGTFGSKDVAYFYNFSIK

SEQ ID NO: 28 - protein, 795 aa, synthetic construct
MNKNNTKLSTRALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLHIYLPKITSMLSDVMKQNYALSLQIEYLSKQLQEISDKLDIINVNVLINSTLTEITPAYQRIKYVNEKFEELTFATETNLKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYIKTKGSDEDAEVIIQAEPGHALVGFEMINDPSPALKVYQAKLKPNYQVDKQSLSETVYGDMDKILCPDKSQQMYYLHNITFPNEYVITEIIFTKKKNSLRYEVIANYYEFSSGDIDLNKKLVKSSEAEYSTLSVSNDAIYMPLGVISETFLTPIKGFGLTVDESSRLVTLTCKSYLREILLATDLSNKATKLIVPPNGFISNLVENGDIEADNIEPWKGNNKNAYVDHTGGVNGTKALYTQDDGEFSQFIGDKLKSKTEYIIQYTVKGNTSIYLKDKKNENVIYEDKNNNLEAFQTITKRFTTELDSSDVYLVFKCKNGYKAWGDNFLITEIRPKEVVSPELIKVENWIGMGGSNHVNPDSLLLFTGGRSILKQNLQLDSYSTYRVRFSLMVIGKAKVIIRNSSEVLFEKSYVNDSEGVLEGVSETFTTKSIQDNFYVELSNEGTFGSKDVAYFYNFSIK

SEQ ID NO: 29 - protein, 803 aa, synthetic construct
MNMNNTKLNARGLQSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLNIYLPKITSMLSDVMKQNYALSLQIEYLSRQLQEISDKLDVINLNVLINSTLTEITPSYQRIKYVNEKFDKLTFATESTLRAKQGIFNEDSFDNNTLENLTDLAELAKSITKNDVDSFEFYLHTFHDVLIGNNLFGRSALKTASELITKDEIKTSGSEIGKVYSFLIVLTSLQAKAFLTLTTCRKLLGLSDIDYTSIMNEHLNNEKNEFRDNILPALSNKFSNPSYAKTIGSDNYAKVILESEPGYALVGFEIINDPIPVLKAYKAKLKQNYQVDNQSLSEIVYLDIDKLFCPENSEQKYYTKNLTFPDGYVITKITFEKKLNNLIYEATANFYDPSTGDIDLNKKQVESTFPQTDYITMDIGDDDGIYMPLGVISETFLTPINSFGLEVDAKSKTLTLKCKSYLREYLLESDLKNKETGLIAPPNVFISNVVKNWDIEEDSLEPWVANNKNAYVDNTGGIERSKALFTQGDGEFSQFIGDKLKPNTDYIIQYTVKGKPAIYLKNKSTGYITYEDTNGNSEEFQTIAVKFTSETDLSQTHLVFKSQNGYEAWGDNFIILEAKLFETPESPELIKFNDWERFGTTYITGNELRIDHSRGGYFRQSLNIDSYSTYDLSFSFSGLWAKVIVKNSRGVVLFEKVKNNGSSYEDISESFTTASNKDGFFIELTAERTSSTFHSFRDISIKEKIE

SEQ ID NO: 30 - protein, 803 aa, synthetic construct
MNMNNTKLNARALPSFIDYFNGIYGFATGIKDIMNMIFKTDTGGDLTLDEILKNQQLLNEISGKLDGVNGSLNDLIAQGNLNTELSKEILKIANEQNQVLNDVNNKLDAINTMLNIYLPKITSMLSDVMKQNYALSLQIEYLSRQLQEISDKLDVINLNVLINSTLTEITPSYQRIKYVNEKFDKLTFATESTLRAKQGIFNEDSFDNNTLENLTDLAELAKSITKNDVDSFEFYLHTFHDVLIGNNLFGRSALKTASELITKDEIKTSGSEIGKVYSFLIVLTSLQAKAFLTLTTCRKLLGLSDIDYTSIMNEHLNNEKNEFRDNILPALSNKFSNPSYAKTIGSDNYAKVILESEPGYALVGFEIINDPIPVLKAYKAKLKQNYQVDNQSLSEIVYLDIDKLFCPENSEQKYYTKNLTFPDGYVITKITFEKKLNNLIYEATANFYDPSTGDIDLNKKQVESTFPQTDYITMDIGDDDGIYMPLGVISETFLTPINSFGLEVDAKSKTLTLKCKSYLREYLLESDLKNKETGLIAPPNVFISNVVKNWDIEEDSLEPWVANNKNAYVDNTGGIERSKALFTQGDGEFSQFIGDKLKPNTDYIIQYTVKGKPAIYLKNKSTGYITYEDTNGNSEEFQTIAVKFTSETDLSQTHLVFKSQNGYEAWGDNFIILEAKLFETPESPELIKFNDWERFGTTYITGNELRIDHSRGGYFRQSLNIDSYSTYDLSFSFSGLWAKVIVKNSRGVVLFEKVKNNGSSYEDISESFTTASNKDGFFIELTAERTSSTFHSFRDISIKEKIE

SEQ ID NO: 31 - DNA, 2334 bp, synthetic construct
  source 1..2334: mol_type = other DNA
  CDS 1..2334: protein_id = 11; transl_table = 1; translation = MLQSFIDYFNGIYGFATGIKDIMNMIFKTDTGGDLTLDEILKNQ QLLNDISGKLDGVNGSLNDLIAQGNLNTELSKEILKIANEQNQVLNDVNNKLDAINTM LRVYLPKITSMLSDVIKQNYALSLQIEYLSKQLQEISDKLDIINVNVLINSTLTEITP AYQRIKYVNEKFEELTFATETSSKVKKDGSPADILDELTELTELAKSVTKNDVDGFEF YLNTFHDVMVGNNLFGRSALKTASELITKENVKTSGSEVGNVYNFLIVLTALQAQAFL TLTTCRKLLGLADIDYTSIMNEHLNKEKEEFRVNILPTLSNTFSNPNYAKVKGSDEDA KMIVEAKPGHALIGFEISNDSITVLKVYEAKLKQNYQVDKDSLSEVIYGDMDKLLCPD QSEQIYYTNNIVFPNEYVITKIDFTKKMKTLRYEVTANFYDSSTGEIDLNKKKVESSE AEYRTLSANDDGVYMPLGVISETFLTPINGFGLQADENSRLITLTCKSYLRELLLATD LSNKETKLIVPPSGFISNIVENGSIEEDNLEPWKANNKNAYVDHTGGVNGTKALYVHK DGGISQFIGDKLKPKTEYVIQYTVKGKPSIHLKDENTGYIHYEDTNNNLEDYQTINKR FTTGTDLKGVYLILKSQNGDEAWGDNFIILEISPSEKLLSPELINTNNWTSTGSTNIS GNTLTLYQGGRGILKQNLQLDSFSTYRVYFSVSGDANVRIRNSREVLFEKRYMSGAKD VSEMFTTKFEKDNFYIELSQGNNLYGGPIVHFYDVSIK
atgctgcagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 32 - DNA, 2367 bp, synthetic construct
  source 1..2367: mol_type = other DNA
  CDS 1..2367: protein_id = 13; transl_table = 1; translation = MNKNNTKLSTRGLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVMKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacaccaagttgtctaccaggggcctccaatccttcatcgactacttcaacggcatctacgggttcgccaccggcatcaaggacatcatgaacatgatcttcaagaccgacaccggcggggacctgaccttggacgagatcttgaagaaccagcagttgctcaacgacatctctggcaagctcgacggcgtgaacgggtccctgaacgacttgatcgcccaggggaacttgaacaccgagctcagcaaggagatcttgaagatcgccaacgagcagaaccaggtgttgaacgacgtcaacaacaagctcgacgccatcaacaccatgctgagggtgtacttgcccaagatcaccagcatgttgtctgacgtcatgaagcagaactacgccctctctctgcagatcgagtacctctccaagcagctgcaggagatcagcgacaagctcgacatcatcaacgtgaacgtcctgatcaactccaccttgaccgagatcacccccgcctaccagaggatcaagtatgtgaacgagaagttcgaggagttgaccttcgccaccgagacttccagcaaggtgaagaaggacggctctccagccgacatcctcgacgagctgaccgagttgaccgagctcgccaagtccgtgaccaagaacgacgtcgacgggttcgagttctacttgaacaccttccacgacgtgatggtcggcaacaacctcttcgggaggagcgccctgaagaccgcctctgagttgatcaccaaggagaacgtgaagacctccggcagcgaggtcgggaacgtctacaacttcctgatcgtgttgaccgccctccaggcccaggccttcttgaccctcaccacctgcaggaagctgttgggcctggccgacatcgactacaccagcatcatgaacgagcacctcaacaaggagaaagaggagttcagggtgaacatcctgccaaccttgagcaacaccttctctaaccctaactacgccaaggtgaaggggtccgacgaggacgccaagatgatcgtcgaggccaagccaggccacgccttgatcgggttcgagatctccaacgacagcatcaccgtgctgaaggtctacgaggccaagttgaagcagaactaccaggtggacaaggactctctctccgaggtcatctacggcgacatggacaagctcctgtgcccagaccagtccgagcagatctactacaccaacaacatcgtgttccctaacgagtatgtgatcaccaagatcgacttcaccaagaagatgaagaccttgcgctacgaggtgaccgccaacttctacgactcttccaccggggagatcgacttgaacaagaagaaggtggagagctctgaggccgagtacaggaccctctccgccaacgacgacggcgtgtacatgcccttgggggtcatcagcgagactttcttgaccccaatcaacggcttcgggctgcaggccgacgagaacagcaggctcatcaccctgacctgcaagtcttacttgagggagttgctcctggccaccgacctcagcaacaaggagactaagctgatcgtgcccccatccggcttcatcagcaacatcgtcgagaacgggtccatcgaggaagacaacttggagccttggaaggccaacaacaagaacgcctacgtggaccacaccggcggggtcaacggcaccaaggccctctacgtgcacaaggacggcggaatcagccagttcatcggggacaagctcaagcctaagaccgagtatgtgatccagtacaccgtcaagggcaagccctccatccacttgaaggacgagaacaccgggtacatccactacgaggacaccaacaacaacttggaggactaccagaccatcaacaagaggttcaccaccggcaccgacctcaagggggtgtacctgatcttgaagtcccagaacggcgacgaggcctggggcgacaacttcatcatcttggagatcagcccttctgagaagttgctcagccccgagctcatcaacaccaacaactggacctctaccggctccaccaacatctccgggaacaccctcaccctgtaccagggcgggaggggcatcttgaagcagaacttgcagctcgactccttcagcacctacagggtgtacttctctgtctccggcgacgccaacgtgaggatcaggaactccagggaggtcttgttcgagaagaggtacatgtccggggccaaggacgtgagcgagatgttcaccaccaagttcgagaaggacaacttctacatcgagctcagccaggggaacaacctgtacggcggacccatcgtgcacttctacgacgtctctatcaag

SEQ ID NO: 33 - DNA, 2367 bp, synthetic construct
  source 1..2367: mol_type = other DNA
  CDS 1..2367: protein_id = 15; transl_table = 1; translation = MNKNNTKLSTRGLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVMKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAKAFLTLTTCRKLLGLADIDYTSIMNEHLNKEKEEFRVNILPTLSNTFSNPN YAKVKGSDEDAKMIVEAKPGHALVGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISEAFLTPINGFGLQADENSRLITLTCK SYLRELLLATDLSNKETKLIVPPSGFIKNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTITKRFTTGTDLKGVYLILKSQNGDEAWEDNFIILEISPSEKLLSPELINTN NWTSTGSTNISGNTLTLYQGGRGILKQNLQLDSFSTYRVYFSVSGDANVRIRNSREVL FEKRYMSGAKDVSEIFTTKLGKDNFYIELSQGNNLNGGPIVKFSDVSIK
atgaacaagaacaacacgaagctgtccacgaggggcctgcaatccttcatcgactacttcaacggcatctacggcttcgccacgggcatcaaggacatcatgaacatgatcttcaagacggacaccggcggcgacctgacgctcgacgagatcctgaagaaccagcagctgctcaacgacatcagcggcaagctcgacggcgtgaacggcagcctgaacgacctcatcgcccagggcaacctcaacacggagctgtccaaggagatcctcaagatcgcgaacgagcagaaccaggtgctgaacgacgtcaacaacaagctcgacgccatcaacaccatgctgagggtgtacctccccaagatcacgtccatgctgtccgacgtcatgaagcagaactacgccctgtccctccagatcgagtacctgtccaagcagctccaggagatcagcgacaagctcgacatcatcaacgtgaacgtcctcatcaacagcacgctgacggagatcacgcccgcgtaccagcgcatcaagtatgtgaacgagaagttcgaggagctgacgttcgccacggagacgtccagcaaggtcaagaaggacggctccccggccgacatcctcgacgagctgaccgagctgacggagctggccaagtccgtgaccaagaacgacgtggacggcttcgagttctacctcaacacgttccacgacgtcatggtgggcaacaacctcttcggccgctccgccctcaagaccgcctccgagctgatcacgaaggagaacgtcaagaccagcggcagcgaggtcggcaacgtctacaacttcctgatcgtgctgaccgccctccaggccaaggcgttcctgaccctgacgacctgccgcaagctcctgggcctcgccgacatcgactacacgagcatcatgaacgagcacctcaacaaggagaaggaggagttcagggtcaacatcctgccgaccctctccaacaccttcagcaacccgaactacgccaaggtcaagggcagcgacgaggacgccaagatgatcgtcgaggcgaagccgggccacgcgctcgtgggcttcgagatcagcaacgacagcatcaccgtgctcaaggtctacgaggcgaagctcaagcagaactaccaggtggacaaggacagcctgtccgaggtcatctacggcgacatggacaagctgctctgcccggaccagtccgagcagatctactacacgaacaacatcgtcttcccgaacgagtatgtgatcacgaagatcgacttcaccaagaagatgaaaaccctccgctacgaggtaaccgcgaacttctacgactccagcaccggcgagatcgacctgaacaagaagaaggtcgagtccagcgaggccgagtacaggacgctgtccgccaacgacgacggcgtgtacatgccgctcggcgtgatcagcgaggcgttcctcacgccgatcaacggcttcggcctccaggccgacgagaacagcaggctcatcaccctgacgtgcaagagctacctgagggagctgctcctggccacggacctgtccaacaaggagacgaagctgatcgtcccgccgtccggcttcatcaagaacatcgtcgagaacggctccatcgaggaggacaacctggagccgtggaaggccaacaacaagaacgcctacgtggaccacaccggcggcgtgaacggcacgaaggccctctacgtgcacaaggacggcggcatcagccagttcatcggcgacaagctcaagcccaagaccgagtatgtcatccagtacaccgtcaagggcaagccctccatccacctgaaggacgagaacacgggctacatccactacgaggacaccaacaacaacctggaggactaccagaccatcaccaagaggttcacgaccggcaccgacctcaagggcgtctacctgatcctcaagtcccagaacggcgacgaggcgtgggaggacaacttcatcatcctggagatcagcccctccgagaagctcctgagcccggagctgatcaacacgaacaactggacgtccaccggctccaccaacatctccggcaacaccctgacgctctaccagggcggcaggggcatcctgaagcagaacctccagctggactccttcagcacctacagggtgtacttcagcgtgtccggcgacgccaacgtccgcatccgcaactcccgcgaggtcctgttcgagaagcgctacatgtccggcgccaaggacgtcagcgagatcttcaccacgaagctcggcaaggacaacttctacatcgagctgtcccagggcaacaacctgaacggcggcccgatcgtcaagttctccgacgtgtccatcaag

SEQ ID NO: 34 - DNA, 2367 bp, synthetic construct
  source 1..2367: mol_type = other DNA
  CDS 1..2367: protein_id = 17; transl_table = 1; translation = MNKNNTKLSTRGLQSFIDYFNGIYGFATGIKDIMNMIFKTDTGG NLTLDEILKNQQLLNEISGKLDGVNGSLNDLIAQGNLNTELSKEILKIANEQNQVLND VNNKLDAINTMLHIYLPKITSMLSDVMKQNYALSLQIEYLSKQLQEISDKLDIINVNV LINSTLTEITPAYQRIKYVNEKFEELTFATETTLKVKKDSSPADILDELTELTELAKS VTKNDVDGFEFYLNTFHDVMVGNNLFGRSALKTASELIAKENVKTSGSEVGNVYNFLI VLTALQAKAFLTLTTCRKLLGLAGIDYTSIMNEHLNKEKEEFRVNILPTLSNTFSNPN YAKVKGSDEDAKMIVEAKPGHALVGFEMSNDSITVLKVYEAKLKQNYQVDKDSLSEVI YGDTDKLFCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacgcgcggcctccaaagcttcatcgactacttcaacggcatctacggcttcgccaccggcatcaaggacatcatgaacatgatcttcaagaccgacacgggcggcaacctcaccctggacgagatcctgaagaaccagcagctgctcaacgagatcagcggcaagctcgacggcgtgaacggctccctcaacgacctgatcgcccagggcaacctcaacaccgagctgtccaaggagatcctcaagatcgccaacgagcagaaccaggtgctcaacgacgtgaacaacaagctggacgcgatcaacaccatgctgcacatctacctgccgaagatcaccagcatgctctccgacgtcatgaagcagaactacgccctgagcctccagatcgagtacctgtccaagcagctccaggagatcagcgacaagctcgacatcatcaacgtgaacgtcctcatcaactccacgctcaccgagatcacgccggcctaccagcgcatcaagtatgtgaacgagaagttcgaggagctgaccttcgccaccgagacgaccctcaaggtgaagaaggactccagcccggccgacatcctggacgagctgaccgagctgacggagctggcgaagtccgtgaccaagaacgacgtggacggcttcgagttctacctgaacaccttccacgacgtcatggtgggcaacaacctgttcggccgctccgcgctcaagaccgcgtccgagctgatcgccaaggagaacgtcaagaccagcggctccgaggtcggcaacgtgtacaacttcctcatcgtgctgaccgccctccaggccaaggcgttcctcaccctcaccacgtgcaggaagctcctgggcctggccggcatcgactacacctccatcatgaacgagcacctgaacaaggagaaggaggagttccgcgtcaacatcctgccgaccctcagcaacaccttcagcaacccgaactacgcgaaggtcaagggcagcgacgaggacgccaagatgatcgtcgaggcgaagccgggccacgccctggtcggcttcgagatgagcaacgactccatcacggtcctcaaggtgtacgaggccaagctcaagcagaactaccaggtggacaaggacagcctgtccgaggtcatctacggcgacacggacaagctgttctgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 35 - DNA, 2367 bp, synthetic construct
  source 1..2367: mol_type = other DNA
  CDS 1..2367: protein_id = 19; transl_table = 1; translation = MNMNNTKLNARGLQSFIDYFNGIYGFATGIKDIMNMIFKTDTGG NLTLDEILKNQQLLNEISGKLDGVNGSLNDLIAQGNLNTELSKEILKIANEQNQVLND VNNKLDAINTMLHIYLPKITSMLSDVMKQNYALSLQIEYLSKQLQEISDKLDIINVNV LINSTLTEITPAYQRIKYVNEKFEELTFATETTLKVKKDSSPADILDELTELTELAKS VTKNDVDGFEFYLNTFHDVMVGNNLFGRSALKTASELIAKENVKTSGSEVGNVYNFLI VLTALQAKAFLTLTTCRKLLGLADIDYTSIMNEHLNKEKEEFRVNILPTLSNTFSNPN YAKVKGSDEDAKMIVEAKPGHALVGFEMSNDSITVLKVYEAKLKQNYQVDKDSLSEVI YGDMDKLLCPDQSEQIYYTNNIVFPNEYVITKIDFTKKMKTLRYEVTANSYDSSTGEI DLNKKKVESSEAEYRTLSAKDDGVYMPLGVISETFLTPINGFGLQADENSRLITLTCK SYLRELLLATDLSNKETKLIVPPSGFISNIVENGNLEGENLEPWIANNKNAYVDHTGG VNGTRALYVHKDGGFSQFIGDKLKPKTEYVIQYTVKGKPSIHLKNENTGYIHYEDTNN NLEDYQTITKRFTTGTDLKGVYLILKSQNGDEAWGDNFTILEISPSEKLLSPELINVN NWIRTGSTHISGNTLTLYQGGGGNLKQNLQLDSFSTYRVNFSVTGDANVRIRNSREVL FEKRYMSGAKDVSEIFTTKLGKDNFYIELSQGNNLYGGPLVKFNDVSIK
atgaacatgaacaacaccaagctcaacgcgaggggcctccaatccttcatcgactacttcaacggcatctacggcttcgccaccggcatcaaggacatcatgaacatgatcttcaagaccgacacgggcggcaacctcaccctcgacgagatcctcaagaaccagcagctgctcaacgagatcagcggcaagctggacggcgtgaacggcagcctgaacgacctcatcgcccagggcaacctgaacaccgagctgtccaaggagatcctgaagatcgccaacgagcagaaccaggtcctgaacgacgtgaacaacaagctggacgccatcaacaccatgctgcacatctacctccccaagatcacgtccatgctgtccgacgtcatgaagcagaactacgcgctgtccctccagatcgagtacctgagcaagcagctccaggagatctccgacaagctggacatcatcaacgtcaacgtgctcatcaacagcacgctcaccgagatcacgccggcgtaccagaggatcaagtatgtcaacgagaagttcgaggagctgaccttcgcgacggagacgaccctcaaggtgaagaaggactccagcccggccgacatcctcgacgagctgaccgagctgacggagctagcgaagtccgtcacgaagaacgacgtggacggcttcgagttctacctcaacaccttccacgacgtcatggtgggcaacaacctcttcggccgctccgcgctcaagaccgcgtccgagctgatcgccaaggagaacgtcaagacgagcggcagcgaggtgggcaacgtctacaacttcctgatcgtgctgacggccctccaggcgaaggcgttcctgaccctcacgacgtgcaggaagctcctcggcctggccgacatcgactacacgagcatcatgaacgagcacctcaacaaggagaaggaggagttccgcgtgaacatcctgccgaccctgtccaacacgttctccaacccgaactacgcgaaggtgaagggcagcgacgaggacgccaagatgatcgtggaggcgaagccgggccacgcgctcgtgggcttcgagatgagcaacgactccatcaccgtgctcaaggtctacgaggccaagctgaagcagaactaccaggtggacaaggacagcctctccgaggtcatctacggcgacatggacaagctcctgtgccccgaccagagcgagcagatctactacaccaacaacatcgtgttccccaacgagtatgtcatcacgaagatcgacttcaccaagaagatgaaaaccctccgctacgaggtgacggcgaacagctacgactccagcaccggcgagatcgacctgaacaagaagaaggtggagtcctccgaggcggagtacaggaccctgtccgccaaggacgacggcgtctacatgcccctgggcgtcatcagcgagaccttcctgacgccgatcaacggcttcggcctccaggcggacgagaactcccgcctcatcacgctgacctgcaagagctacctcagggagctgctgctcgcgacggacctgtccaacaaggagacgaagctcatcgtgccgcccagcggcttcatcagcaacatcgtggagaacggcaacctggagggcgagaacctggagccctggatcgcgaacaacaagaacgcctacgtggaccacacgggcggcgtgaacggcacgcgcgccctgtatgtccacaaggacggcggcttctcccagttcatcggcgacaagctcaagccgaaaaccgagtatgtgatccagtacacggtgaagggcaagccgtccatccacctcaagaacgagaacacgggctacatccactacgaggacacgaacaacaacctcgaagactaccagaccatcacgaagcgcttcacgaccggcacggacctgaagggcgtctacctcatcctgaagtcccagaacggcgacgaggcgtggggcgacaacttcaccatcctggagatcagcccgtccgagaagctcctgagccccgagctgatcaacgtgaacaactggatcaggacgggcagcacccacatctccggcaacaccctgaccctctaccagggcggcggcggcaacctgaagcagaacctccagctcgacagcttctccacctaccgcgtgaacttctccgtgaccggcgacgcgaacgtccgcatcaggaactccagggaggtcctcttcgagaagaggtacatgtccggcgcgaaggacgtgtccgagatcttcacgaccaagctcggcaaggacaacttctacatcgagctgtcccagggcaacaacctgtacggcggcccgctggtgaagttcaacgacgtcagcatcaag

SEQ ID NO: 36 - DNA, 2367 bp, synthetic construct
  source 1..2367: mol_type = other DNA
  CDS 1..2367: protein_id = 21; transl_table = 1; translation = MNMNNTKLNARGLQSFIDYFNGIYGFATGIKDIMNMIFKTDTGG NLTLDEILKNQQLLNEISGKLDGVNGSLNDLIAQGNLNTELSKEILKIANEQNQVLND VNNKLDAINTMLHIYLPKITSMLSDVMKQNYALSLQIEYLSKQLQEISDKLDIINVNV LINSTLTEITPAYQRIKYVNEKFEELTFATETTLKVKKDSSPADILDELTELTELAKS VTKNDVDGFEFYLNTFHDVMVGNNLFGRSALKTASELIAKENVKTSGSEVGNVYNFLI VLTALQAKAFLTLTTCRKLLGLADIDYTSIMNEHLNKEKEEFRVNILPTLSNTFSNPN YAKVKGSDEDAKMIVEAKPGHALVGFEMSNDSITVLKVYEAKLKQNYQVDKDSLSEVI YGDMDKLLCPDQSEQIYYTNNIVFPNEYVITKIDFTKKMKTLRYEVTANSYDSSTGEI DLNKKKVESSEAEYRTLSAKDDGVYMPLGVISETFLTPINGFGLQADENSRLITLTCK SYLRELLLATDLSNKETKLIVPPSGFISNIVENGNLEGENLEPWIANNKNAYVDHTGG VNGTRALYVHKDGGFSQFIGDKLKPKTEYVIQYTVKGKPSIHLKNENTGYIHYEDTNN NLEDYQTITKRFTTGTDLKGVYLILKSQNGDEAWGDNFTILEISPSEKLLSPELINVN NWIRTGSTHISGNTLTLYQGGGGNLKQNLQLDSFSTYRVNFSVTGDANVRIRNSREVL FEKRYMSGAKDVSEIFTTKLGKDNPYIELSQGNNLYGGPLVKPNDVSIK
atgaacatgaacaacacgaagctgaacgcccgcggcctccaaagcttcatcgactacttcaacggcatctacggcttcgcgacgggcatcaaggacatcatgaacatgatcttcaagacggacaccggcggcaacctcacgctggacgagatcctgaagaaccagcagctgctgaacgagatctccggcaagctggacggcgtgaacggctccctgaacgacctgatcgcccagggcaacctcaacaccgagctgtccaaggagatcctcaagatcgcgaacgagcagaaccaggtgctcaacgacgtcaacaacaagctcgacgccatcaacaccatgctgcacatctacctgcccaagatcacgagcatgctgtccgacgtgatgaagcagaactacgcgctgtccctccagatcgagtacctcagcaagcagctccaggagatcagcgacaagctggacatcatcaacgtcaacgtgctgatcaacagcaccctgacggagatcacgccggcctaccagaggatcaagtatgtgaacgagaagttcgaggagctgaccttcgccacggagacgaccctgaaggtgaagaaggactccagcccggccgacatcctcgacgagctgacggagctgaccgagctggccaagagcgtcaccaagaacgacgtggacggcttcgagttctacctgaacacgttccacgacgtgatggtcggcaacaacctcttcggccgctccgcgctcaagaccgccagcgagctgatcgccaaggagaacgtcaagaccagcggcagcgaggtcggcaacgtctacaacttcctgatcgtgctgaccgccctccaggcgaaggcgttcctcacgctgaccacgtgcaggaagctcctcggcctggccgacatcgactacaccagcatcatgaacgagcacctgaacaaggagaaggaggagttcagggtgaacatcctgccgacgctgtccaacaccttctccaacccgaactacgccaaggtgaagggctccgacgaggacgccaagatgatcgtggaggccaagccgggccacgcgctcgtgggcttcgagatgtccaacgactccatcaccgtcctcaaggtgtacgaggccaagctgaagcagaactaccaggtggacaaggacagcctgagcgaggtcatctacggcgacatggacaagctcctctgcccggaccagtccgagcagatctactacaccaacaacatcgtgttccccaacgagtatgtgatcacgaagatcgacttcaccaagaagatgaaaaccctccgctacgaggtgacggccaactcctacgactccagcacgggcgagatcgacctcaacaagaagaaggtcgagtcctccgaggccgagtaccgcaccctgagcgccaaggacgacggcgtgtacatgccgctgggcgtcatcagcgagacgttcctgaccccgatcaacggcttcggcctccaggccgacgagaacagcaggctgatcacgctcacctgcaagtcctacctccgcgagctgctgctcgcgaccgacctcagcaacaaggagacgaagctcatcgtgccgcccagcggcttcatcagcaacatcgtggagaacggcaacctcgaaggcgagaacctggagccctggatcgccaacaacaagaacgcctacgtggaccacacgggcggcgtcaacggcacgagggcgctctacgtccacaaggacggcggcttctcccagttcatcggcgacaagctgaagcccaagacggagtatgtgatccagtacaccgtcaagggcaagccgtccatccacctgaagaacgagaacaccggctacatccactacgaggacaccaacaacaacctggaggactaccagacgatcaccaagcgcttcaccacgggcaccgacctcaagggcgtctacctcatcctgaagtcccagaacggcgacgaggcgtggggcgacaacttcacgatcctggagatcagcccgtccgagaagctgctctcccccgagctgatcaacgtgaacaactggatcaggaccggctccacgcacatctccggcaacacgctgacgctctaccagggcggcggcggcaacctcaagcagaacctccagctcgacagcttctccacctacagggtgaacttctccgtgacgggcgacgccaacgtgcgcatccgcaactcccgcgaggtgctcttcgagaagcgctacatgagcggcgccaaggacgtctccgagatcttcacgaccaagctgggcaaggacaacccctacatcgagctgtcccagggcaacaacctgtacggcggcccgctggtgaagccgaacgacgtgtccatcaag

SEQ ID NO: 37 - DNA, 2364 bp, synthetic construct
  source 1..2364: mol_type = other DNA
  CDS 1..2364: protein_id = 23; transl_table = 1; translation = MNKNNTKLSTRGLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVMKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAKAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDKFTILEIKPAEDLLSPELINPN SWITTPGASISGNKLFINLGTNGTFRQSLSLNSYSTYSISFTASGPFNVTVRNSRGVL FERSNLMSSTSHISGTFKTESNNTGLYVELSRRSGGGGHISFENVSIK
atgaacaagaacaacacgaagctctccacccgcggcctgcaaagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatgaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctaaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaagttcaccatcctggagatcaagccggccgaggacctcctgtcccccgagctgatcaacccgaactcctggatcacgacccccggcgccagcatctccggcaacaagctcttcatcaacctcggcaccaacggcaccttcaggcagagcctctccctgaacagctactccacctacagcatctccttcaccgcctccggcccgttcaacgtgacggtcaggaactccaggggcgtgctcttcgagaggtccaacctgatgagcagcacctcccacatcagcggcacgttcaagaccgagtccaacaacaccggcctctacgtcgagctgtccaggcgctccggcggcggcggccacatctccttcgagaacgtgtccatcaag

SEQ ID NO: 38 - DNA, 2358 bp, synthetic construct
  source 1..2358: mol_type = other DNA
  CDS 1..2358: protein_id = 25; transl_table = 1; translation = MNMNNAKLNARGLQSFIDYFNGIYGFAIGIKDIMNMIFKTDTGS NLTLDEILKNQQLLNEISGKLDGVNGSLNDLIAQGNLNTELAKQILKVANEQNQVLND VNNKLDTINSMLKIYLPKITSMLSDVMKQNYVLSLQIEYLSKQLQEISDKLDIINVNV LINSTLTEITPAYQRMKYVNEKFEELTFATETTLKVKKDSPPADILDELTELTELAKS VTKNDVDGFEFYLNTFHDVMVGNNLFGRSALKTASELIAKENVKTSGSEVGNVYNFLI VLTALQAKAFLTLTTCRKLLGLADIDYTSIMNEHLNKEKEEFRVNILPTLSNTFSNPN YAKAKGSNEDTKMIVEAKPGYVLVGFEMSNDSITVLKAYQAKLKKDYQIDKDSLSEII YSDTDKLLCPDQSEQIYYTKNIAFPNEYVITKIAFTKKMNSLRYEATANFYDSSTGDI DLNKTKVESSEAEYSMLKASDDEVYMPLGLISETFLNPINGFRLAVDENSRLVTLTCR SYLRETLLATDLNNKETKLIVPPNVFISNIVENGNIEMDTLEPWKANNENANVDYSGG VNGTRALYVHKDGEFSHFIGDKLKSKTEYLIRYIVKGKASIFLKDEKNENYIYEDTNN NLEDYQTITKRFTTGTDSTGVYLIFNSQNGDEAWGDNFIILEISPCEKLLSPELIKTD KWNSTGSTYISDDRLTLYRGGRGILKQNLQLDGFSTYRVNFSVDGDANVRIRNSREVL LEKRYLNRKGVSEMFTTKFDKDNFYVELSQGDNLGTIVHFYDFSIK
atgaacatgaataatgcaaagctcaatgccaggggccttcaatcttttatagattacttcaatggaatctacggcttcgctatcggtatcaaggatatcatgaacatgatattcaagactgatactggcagcaacttgacgctcgatgaaattctaaagaaccagcagttgctcaacgagatttctgggaaacttgacggcgtcaacggcagcctcaacgacctcattgcgcagggcaacctcaacacagagttagcgaagcaaatcctcaaggtagctaatgaacaaaatcaagtgcttaacgacgttaacaacaaattggacacgattaactctatgctcaagatctatcttccgaagataacctccatgctttcagatgtgatgaagcaaaactacgtcctctctctacaaatcgaatatctctctaagcagctgcaggagatcagtgataagctggacatcattaacgtgaacgtcctcatcaattcgaccctgacagaaataaccccggcttaccagaggatgaaatatgtcaacgaaaagttcgaggaactcacttttgcgacagaaactacgcttaaagtaaagaaggactcacctcccgccgacatactggatgagctgaccgaactcaccgaacttgcgaagtcagttacaaaaaatgatgtagacggttttgagttctaccttaacaccttccatgatgtcatggtgggcaataatctattcgggcgttccgcacttaagactgcttcggaactgatcgccaaagagaatgtcaagacaagtgggtccgaagttggcaacgtttacaacttcttgatagtcctaactgccctccaggccaaggcgttcttgacactgactacctgtagaaagttgctcggtttagcagatatcgattacacaagtattatgaacgagcacctgaacaaggagaaagaggagttccgcgttaacattttacctactttgtcaaatacattcagtaatccaaattatgcaaaggcgaagggttccaacgaagatacaaagatgattgtggaagccaagccgggctacgtgcttgtggggtttgagatgtcaaatgacagcattaccgttctgaaagcatatcaggccaagttgaagaaggactaccaaatagataaagattcactctcggagataatatattcggatactgacaagttactctgcccggaccagtcagagcagatctactacaccaaaaacatagcttttcctaatgagtatgtaattactaagatcgccttcaccaaaaagatgaactccctgaggtacgaggcaactgccaatttttatgacagcagcacaggcgacatcgacttgaataaaacaaaggttgaatcatccgaagctgagtactctatgctgaaagcaagcgacgacgaggtgtacatgccattgggtcttatttctgagacgttccttaaccccatcaatggattcaggctggccgtggatgagaactccagactcgtcacgttaacatgccggtcgtatctgcgcgagacactgctagccacggatttgaataacaaggaaacaaagttgattgttccaccgaatgttttcataagcaatatcgtggaaaacggtaacatcgagatggacactctggagccgtggaaggcgaacaatgaaaacgccaatgtggactactccggtggagttaatggaacccgtgcgctgtatgtccacaaggatggcgagtttagtcattttattggagacaaactgaagagcaagacggagtatcttataagatatattgtgaaaggcaaagccagcatttttttgaaagacgagaagaatgaaaattacatctatgaggacaccaacaataacttggaggattatcagactattacgaaaaggttcacgactggcaccgattcaacaggggtctacctgatatttaatagccagaacggagacgaggcgtggggcgacaatttcatcatcctggagatttctccttgcgagaagctcctgagcccagagctgattaagacggacaagtggaactcaacaggcagcacctacatctctgatgaccgccttacgctgtacagggggggtcgtggaatcctgaaacaaaatcttcagctggacgggtttagcacataccgcgtcaatttttcggttgatggtgatgcgaatgttcggattcgaaattccagagaggttttgcttgagaaacgctacctgaaccgcaagggcgtcagtgaaatgtttacaactaagttcgacaaagacaacttctatgtggagctaagtcagggagataacctcggcaccatcgtgcacttctacgattttagcatcaaa

SEQ ID NO: 39 - DNA, 2385 bp, synthetic construct
  source 1..2385: mol_type = other DNA
  CDS 1..2385: protein_id = 27; transl_table = 1; translation = MNKNNTKLSTRGLQSFIDYFNGIYGFATGIKDIMNMIFKTDTGG DLTLDEILKNQQLLNEISGKLDGVNGSLNDLIAQGNLNTELSKEILKIANEQNQVLND VNNKLDAINTMLHIYLPKITSMLSDVMKQNYALSLQIEYLSKQLQEISDKLDIINVNV LINSTLTEITPAYQRIKYVNEKFEELTFATETNLKVKKDGSPADILDELTELTELAKS VTKNDVDGFEFYLNTFHDVMVGNNLFGRSALKTASELITKENVKTSGSEVGNVYNFLI VLTALQAKAFLTLTTCRKLLGLADIDYTSIMNEHLNKEKEEFRVNILPTLSNTFSNPN YIKTKGSDEDAEVIIQAEPGHALVGFEMINDPSPALKVYQAKLKPNYQVDKQSLSETV YGDMDKILCPDKSQQMYYLHNITFPNEYVITEIIFTKKKNSLRYEVIANYYEFSSGDI DLNKKLVKSSEAEYSTLSVSNDAIYMPLGVISETFLTPIKGFGLTVDESSRLVTLTCK SYLREILLATDLSNKATKLIVPPNGFISNLVENGDIEADNIEPWKGNNKNAYVDHTGG VNGTKALYTQDDGEFSQFIGDKLKSKTEYIIQYTVKGNTSIYLKDKKNENVIYEDKNN NLEAFQTITKRFTTELDSSDVYLVFKCKNGYKAWGDNFLITEIRPKEVVSPELIKVEN WIGMGGSNHVNPDSLLLFTGGRSILKQNLQLDSYSTYRVRFSLMVIGKAKVIIRNSSE VLFEKSYVNDSEGVLEGVSETFTTKSIQDNFYVELSNEGTFGSKDVAYFYNFSIK
atgaacaagaacaacacgaagctgtccaccaggggcctccaatccttcatcgactacttcaacggcatctacggcttcgcgaccggcatcaaggacatcatgaacatgatcttcaagacggacaccggcggcgacctgacgctcgacgagatcctcaagaaccagcagctgctcaacgagatcagcggcaagctcgacggcgtcaacggctccctcaacgacctgatcgcccagggcaacctgaacacggagctgtccaaggagatcctcaagatcgccaacgagcagaaccaggtcctcaacgacgtcaacaacaagctcgacgccatcaacacgatgctccacatctacctccccaagatcacctccatgctgtccgacgtgatgaagcagaactacgcgctgtccctccagatcgagtacctgagcaagcagctccaggagatctccgacaagctcgacatcatcaacgtgaacgtcctcatcaacagcacgctgaccgagatcaccccggcgtaccagcgcatcaagtatgtgaacgagaagttcgaggagctgaccttcgccaccgagacgaacctcaaggtgaagaaggacggctccccggcggacatcctcgacgagctgaccgagctgacggagctggccaagagcgtgaccaagaacgacgtggacggcttcgagttctacctgaacaccttccacgacgtgatggtcggcaacaacctgttcggcaggagcgcgctcaagaccgcgtccgagctgatcacgaaggagaacgtcaagacgtccggcagcgaggtgggcaacgtctacaacttcctgatcgtcctcaccgcgctgcaagccaaggcgttcctcaccctcaccacgtgcaggaagctcctgggcctggccgacatcgactacaccagcatcatgaacgagcacctgaacaaggagaaggaggagttccgcgtgaacatcctgccgacgctctccaacacgttcagcaacccgaactacatcaagacgaagggctccgacgaggacgccgaggtcatcatccaggcggagcccggccacgcgctggtgggcttcgagatgatcaacgaccccagccccgccctcaaggtctaccaggccaagctcaagccgaactaccaggtggacaagcagtccctgagcgagacggtctacggcgacatggacaagatcctgtgcccggacaagagccagcagatgtactacctccacaacatcaccttcccgaacgagtatgtcatcaccgagatcatcttcaccaagaagaagaactccctccgctacgaggtcatcgccaactactacgagttcagctccggcgacatcgacctcaacaagaagctcgtcaagtccagcgaggccgagtacagcaccctctccgtgagcaacgacgcgatctacatgccgctgggcgtgatcagcgagacgttcctgaccccgatcaagggcttcggcctcaccgtggacgagtcctccaggctcgtgacgctcacctgcaagtcctacctccgcgagatcctgctcgccacggacctctccaacaaggccacgaagctcatcgtgcccccgaacggcttcatctccaacctggtcgagaacggcgacatcgaggcggacaacatcgagccctggaagggcaacaacaagaacgcctacgtggaccacaccggcggcgtgaacggcaccaaggccctctacacccaggacgacggcgagttctcccagttcatcggcgacaagctgaagtccaagacggagtacatcatccagtacaccgtgaagggcaacacctccatctacctgaaggacaagaagaacgagaacgtgatctacgaggacaagaacaacaacctggaggcgttccagaccatcacgaagcgcttcaccaccgagctggactcctccgacgtctacctcgtgttcaagtgcaagaacggctacaaggcgtggggcgacaacttcctcatcacggagatcaggccgaaggaggtcgtcagcccggagctgatcaaggtggagaactggatcggcatgggcggctccaaccacgtgaaccccgacagcctcctcctgttcaccggcggcaggagcatcctcaagcagaacctccagctggactcctacagcacgtacagggtccgcttctccctcatggtcatcggcaaggcgaaggtaatcatccgcaactcctccgaggtcctcttcgagaagagctacgtcaacgacagcgagggcgtcctcgaaggcgtcagcgagaccttcaccaccaagtccatccaggacaacttctacgtcgagctgtccaacgagggcacgttcggctccaaggacgtcgcctacttctacaacttctccatcaag

SEQ ID NO: 40 - DNA, 2409 bp, synthetic construct
  source 1..2409: mol_type = other DNA
  CDS 1..2409: protein_id = 29; transl_table = 1; translation = MNMNNTKLNARGLQSFIDYFNGIYGFATGIKDIMNMIFKTDTGG DLTLDEILKNQQLLNEISGKLDGVNGSLNDLIAQGNLNTELSKEILKIANEQNQVLND VNNKLDAINTMLNIYLPKITSMLSDVMKQNYALSLQIEYLSRQLQEISDKLDVINLNV LINSTLTEITPSYQRIKYVNEKFDKLTFATESTLRAKQGIFNEDSFDNNTLENLTDLA ELAKSITKNDVDSFEFYLHTFHDVLIGNNLFGRSALKTASELITKDEIKTSGSEIGKV YSFLIVLTSLQAKAFLTLTTCRKLLGLSDIDYTSIMNEHLNNEKNEFRDNILPALSNK FSNPSYAKTIGSDNYAKVILESEPGYALVGFEIINDPIPVLKAYKAKLKQNYQVDNQS LSEIVYLDIDKLFCPENSEQKYYTKNLTFPDGYVITKITFEKKLNNLIYEATANFYDP STGDIDLNKKQVESTFPQTDYITMDIGDDDGIYMPLGVISETFLTPINSFGLEVDAKS KTLTLKCKSYLREYLLESDLKNKETGLIAPPNVFISNVVKNWDIEEDSLEPWVANNKN AYVDNTGGIERSKALFTQGDGEFSQFIGDKLKPNTDYIIQYTVKGKPAIYLKNKSTGY ITYEDTNGNSEEFQTIAVKFTSETDLSQTHLVFKSQNGYEAWGDNFIILEAKLFETPE SPELIKFNDWERFGTTYITGNELRIDHSRGGYFRQSLNIDSYSTYDLSFSFSGLWAKV IVKNSRGVVLFEKVKNNGSSYEDISESFTTASNKDGFFIELTAERTSSTFHSFRDISI KEKIE
atgaacatgaacaacaccaagctcaacgcccgcggcctccaaagcttcatcgactacttcaacggcatctacggcttcgcgaccggcatcaaggacatcatgaacatgatcttcaagaccgacaccggcggcgacctcaccctggacgagatcctgaagaaccagcagctgctcaacgagatctccggcaagctggacggcgtgaacggcagcctcaacgacctgatcgcgcagggcaacctcaacaccgagctgtccaaggagatcctcaagatcgccaacgagcagaaccaggtgctgaacgacgtcaacaacaagctcgacgccatcaacacgatgctgaacatctacctcccgaagatcaccagcatgctgtccgacgtgatgaagcagaactacgcgctgtccctccagatcgagtacctgagccgccagctccaggagatctccgacaagctcgacgtgatcaacctcaacgtcctcatcaactccaccctcacggagatcaccccgagctaccagcgcatcaagtatgtgaacgagaagttcgacaagctcacgttcgcgaccgagtccaccctgcgcgccaagcagggcatcttcaacgaggactccttcgacaacaacaccctggagaacctgaccgacctcgcggagctggccaagagcatcaccaagaacgacgtggactccttcgagttctacctgcacaccttccacgacgtcctcatcggcaacaacctcttcggcaggagcgccctcaagacggcctccgagctgatcaccaaggacgagatcaagacgagcggctccgagatcggcaaggtgtactccttcctgatcgtgctcacctccctccaggccaaggcgttcctgaccctcaccacgtgccgcaagctgctgggcctctccgacatcgactacacgagcatcatgaacgagcacctcaacaacgagaagaacgagttcagggacaacatcctgccggcgctctccaacaagttcagcaaccccagctacgccaagaccatcggctccgacaactacgcgaaggtcatcctggagtccgagcccggctacgcgctcgtgggcttcgagatcatcaacgaccccatcccggtgctcaaggcgtacaaggcgaagctcaagcagaactaccaggtggacaaccagagcctctccgagatcgtgtacctcgacatcgacaagctgttctgccccgagaactccgagcagaagtactacacgaagaacctcacgttcccggacggctacgtgatcaccaagatcaccttcgagaagaagctcaacaacctgatctacgaggcgacggcgaacttctacgacccgtccaccggcgacatcgacctgaacaagaagcaggtggagtccaccttcccgcagaccgactacatcacgatggacatcggcgacgacgacggcatctacatgccgctgggcgtgatcagcgagacgttcctcacgcccatcaactccttcggcctggaggtggacgccaagagcaagacgctgaccctcaagtgcaagagctacctcagggagtacctcctggagagcgacctgaagaacaaggagacgggcctgatcgcgcccccgaacgtgttcatctccaacgtggtcaagaactgggacatcgaggaggacagcctggagccgtgggtcgccaacaacaagaacgcctacgtggacaacaccggcggcatcgagaggagcaaggcgctgttcacgcagggcgacggcgagttctcccagttcatcggcgacaagctcaagccgaacaccgactacatcatccagtacaccgtgaagggcaagccggccatctacctcaagaacaagagcaccggctacatcacgtacgaggacaccaacggcaacagcgaggagttccagaccatcgcggtgaagttcaccagcgagacggacctcagccagacgcacctggtgttcaagtcccagaacggctacgaggcgtggggcgacaacttcatcatcctggaggccaagctgttcgagacgcccgagagcccggagctgatcaagttcaacgactgggagaggttcggcacgacctacatcacgggcaacgagctgaggatcgaccacagcaggggcggctacttcaggcagagcctgaacatcgactcctacagcacctacgacctgtccttcagcttcagcggcctctgggccaaggtcatcgtgaagaactccaggggcgtcgtgctgttcgagaaggtcaagaacaacggctccagctacgaggacatctccgagagcttcaccacggcgagcaacaaggacggcttcttcatcgagctgacggccgagcgcacgagcagcacgttccacagcttcagggacatctccatcaaggagaagatcgag

SEQ ID NO: 41 - protein, 789 aa, synthetic construct
MNKNNTKLSTRPLDSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 42 - protein, 789 aa, synthetic construct
MNKNNTKLSTRGLNSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 43 - protein, 789 aa, synthetic construct
MNKNNTKLSTRGLLSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 44 - protein, 789 aa, synthetic construct
MNKNNTKLSTRGLRSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 45 - protein, 789 aa, synthetic construct
MNKNNTKLSTRYLKSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 46 - protein, 789 aa, synthetic construct
MNKNNTKLSTRSLVSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 47 - protein, 789 aa, synthetic construct
MNKNNTKLSTRHLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 48 - protein, 789 aa, synthetic construct
MNKNNTKLSTRHLDSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 49 - protein, 789 aa, synthetic construct
MNKNNTKLSTRVLCSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 50 - protein, 789 aa, synthetic construct
MNKNNTKLSTRPLGSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 51 - protein, 789 aa, synthetic construct
MNKNNTKLSTRWLTSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 52 - protein, 789 aa, synthetic construct
MNKNNTKLSTRGLDSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 53 - protein, 789 aa, synthetic construct
MNKNNTKLSTRFLISFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 54 - protein, 789 aa, synthetic construct
MNKNNTKLSTRGLASFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 55 - protein, 789 aa, synthetic construct
MNKNNTKLSTRFLHSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 56 - protein, 789 aa, synthetic construct
MNKNNTKLSTRNLQSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 57 - protein, 789 aa, synthetic construct
MNKNNTKLSTRQLHSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 58 - protein, 789 aa, synthetic construct
MNKNNTKLSTRPLMSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 59 - protein, 789 aa, synthetic construct
MNKNNTKLSTRDLFSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 60 - protein, 789 aa, synthetic construct
MNKNNTKLSTRHLKSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 61 - protein, 789 aa, synthetic construct
MNKNNTKLSTRFLMSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 62 - protein, 789 aa, synthetic construct
MNKNNTKLSTRELPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 63 - protein, 789 aa, synthetic construct
MNKNNTKLSTRGLISFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 64 - protein, 789 aa, synthetic construct
MNKNNTKLSTRDLGSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 65 - protein, 789 aa, synthetic construct
MNKNNTKLSTRQLVSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 66 - protein, 789 aa, synthetic construct
MNKNNTKLSTRSLGSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 67 - protein, 789 aa, synthetic construct
MNKNNTKLSTRFLGSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 68 - protein, 789 aa, synthetic construct
MNKNNTKLSTRGLSSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 69 - protein, 789 aa, synthetic construct
MNKNNTKLSTRRLMSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 70 - protein, 789 aa, synthetic construct
MNKNNTKLSTRHLPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 71 - protein, 789 aa, synthetic construct
MNKNNTKLSTRTLPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 72 - protein, 789 aa, synthetic construct
MNKNNTKLSTRALCSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 73 - protein, 789 aa, synthetic construct
MNKNNTKLSTRALVSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 74 - protein, 789 aa, synthetic construct
MNKNNTKLSTRALASFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 75 - protein, 789 aa, synthetic construct
MNKNNTKLSTRALDSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 76 - protein, 789 aa, synthetic construct
MNKNNTKLSTRALESFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 77 - protein, 789 aa, synthetic construct
MNKNNTKLSTRALFSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 78 - protein, 789 aa, synthetic construct
MNKNNTKLSTRALHSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 79 - protein, 789 aa, synthetic construct
MNKNNTKLSTRALISFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 80 - protein, 789 aa, synthetic construct
MNKNNTKLSTRALKSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVIKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 81 - DNA, 2367 bp, synthetic construct
  source 1..2367: mol_type = other DNA
  CDS 1..2367: protein_id = 41; transl_table = 1; translation = MNKNNTKLSTRPLDSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcccgctggacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 82 - DNA, 2367 bp, synthetic construct
  source 1..2367: mol_type = other DNA
  CDS 1..2367: protein_id = 42; transl_table = 1; translation = MNKNNTKLSTRGLNSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgaacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 83 - DNA, 2367 bp, synthetic construct
  source 1..2367: mol_type = other DNA
  CDS 1..2367: protein_id = 43; transl_table = 1; translation = MNKNNTKLSTRGLLSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgctgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 84 - DNA, 2367 bp, synthetic construct
  source 1..2367: mol_type = other DNA
  CDS 1..2367: protein_id = 44; transl_table = 1; translation = MNKNNTKLSTRGLRSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgaggagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 85 - DNA, 2367 bp, synthetic construct
  source 1..2367: mol_type = other DNA
  CDS 1..2367: protein_id = 45; transl_table = 1; translation = MNKNNTKLSTRYLKSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgctacctgaagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 86 - DNA, 2367 bp, synthetic construct
  source 1..2367: mol_type = other DNA
  CDS 1..2367: protein_id = 46; transl_table = 1; translation = MNKNNTKLSTRSLVSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgctccctggtgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 87 - DNA, 2367 bp, synthetic construct
  source 1..2367: mol_type = other DNA
  CDS 1..2367: protein_id = 47; transl_table = 1; translation = MNKNNTKLSTRHLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccacctgcagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 88 - DNA, 2367 bp, synthetic construct
  source 1..2367: mol_type = other DNA
  CDS 1..2367: protein_id = 48; transl_table = 1; translation = MNKNNTKLSTRHLDSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccacctggacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 89 - DNA, 2367 bp, synthetic construct
  source 1..2367: mol_type = other DNA
  CDS 1..2367: protein_id = 49; transl_table = 1; translation = MNKNNTKLSTRVLCSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgtgctgtgcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 90 - DNA, 2367 bp, synthetic construct
  source 1..2367: mol_type = other DNA
  CDS 1..2367: protein_id = 50; transl_table = 1; translation = MNKNNTKLSTRPLGSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcccgctgggcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 91 - DNA, 2367 bp, synthetic construct
  source 1..2367: mol_type = other DNA
  CDS 1..2367: protein_id = 51; transl_table = 1; translation = MNKNNTKLSTRWLTSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgctggctgaccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 92 - DNA, 2367 bp, synthetic construct
  source 1..2367: mol_type = other DNA
  CDS 1..2367: protein_id = 52; transl_table = 1; translation = MNKNNTKLSTRGLDSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctggacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 93 - DNA, 2367 bp, synthetic construct
  source 1..2367: mol_type = other DNA
  CDS 1..2367: protein_id = 53; transl_table = 1; translation = MNKNNTKLSTRFLISFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcttcctgatcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 94 - DNA, 2367 bp, synthetic construct
  source 1..2367: mol_type = other DNA
  CDS 1..2367: protein_id = 54; transl_table = 1; translation = MNKNNTKLSTRGLASFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctggccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 95 - DNA, 2367 bp, synthetic construct
  source 1..2367: mol_type = other DNA
  CDS 1..2367: protein_id = 55; transl_table = 1; translation = MNKNNTKLSTRFLHSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcttcctgcacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 96 - DNA, 2367 bp, synthetic construct
  source 1..2367: mol_type = other DNA
  CDS 1..2367: protein_id = 56; transl_table = 1; translation = MNKNNTKLSTRNLQSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcaacctgcagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 97 - DNA, 2367 bp, synthetic construct
  source 1..2367: mol_type = other DNA
  CDS 1..2367: protein_id = 57; transl_table = 1; translation = MNKNNTKLSTRQLHSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccagctgcacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 98 - DNA, 2367 bp, synthetic construct
  source 1..2367: mol_type = other DNA
  CDS 1..2367: protein_id = 58; transl_table = 1; translation = MNKNNTKLSTRPLMSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcccgctgatgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 99 - DNA, 2367 bp, synthetic construct
  source 1..2367: mol_type = other DNA
  CDS 1..2367: protein_id = 59; transl_table = 1; translation = MNKNNTKLSTRDLFSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgacctgttcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 100 - DNA, 2367 bp, synthetic construct
  source 1..2367: mol_type = other DNA
  CDS 1..2367: protein_id = 60; transl_table = 1; translation = MNKNNTKLSTRHLKSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccacctgaagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 101 - DNA, 2367 bp, synthetic construct
  source 1..2367: mol_type = other DNA
  CDS 1..2367: protein_id = 61; transl_table = 1; translation = MNKNNTKLSTRFLMSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcttcctgatgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 102 - DNA, 2367 bp, synthetic construct
  source 1..2367: mol_type = other DNA
  CDS 1..2367: protein_id = 62; transl_table = 1; translation = MNKNNTKLSTRELPSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgagctgcccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 103 - DNA, 2367 bp, synthetic construct
  source 1..2367: mol_type = other DNA
  CDS 1..2367: protein_id = 63; transl_table = 1; translation = MNKNNTKLSTRGLISFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgatcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 104 - DNA, 2367 bp, synthetic construct
  source 1..2367: mol_type = other DNA
  CDS 1..2367: protein_id = 64; transl_table = 1; translation = MNKNNTKLSTRDLGSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgacctgggcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 105 - DNA, 2367 bp, synthetic construct
  source 1..2367: mol_type = other DNA
  CDS 1..2367: protein_id = 65; transl_table = 1; translation = MNKNNTKLSTRQLVSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccagctggtgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 106 - DNA, 2367 bp, synthetic construct
  source 1..2367: mol_type = other DNA
  CDS 1..2367: protein_id = 66; transl_table = 1; translation = MNKNNTKLSTRSLGSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgctccctgggcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 107 - DNA, 2367 bp, synthetic construct
  source 1..2367: mol_type = other DNA
  CDS 1..2367: protein_id = 67; transl_table = 1; translation = MNKNNTKLSTRFLGSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcttcctgggcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 108 - DNA, 2367 bp, synthetic construct
  source 1..2367: mol_type = other DNA
  CDS 1..2367: protein_id = 68; transl_table = 1; translation = MNKNNTKLSTRGLSSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcggcctgtccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 109 - DNA, 2367 bp, synthetic construct
  source 1..2367: mol_type = other DNA
  CDS 1..2367: protein_id = 69; transl_table = 1; translation = MNKNNTKLSTRRLMSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcaggctgatgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 110 - DNA, 2367 bp, synthetic construct
  source 1..2367: mol_type = other DNA
  CDS 1..2367: protein_id = 70; transl_table = 1; translation = MNKNNTKLSTRHLPSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgccacctgcccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 111 - DNA, 2367 bp, synthetic construct
  source 1..2367: mol_type = other DNA
  CDS 1..2367: protein_id = 71; transl_table = 1; translation = MNKNNTKLSTRTLPSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcaccctgcccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 112 - DNA, 2367 bp, synthetic construct
  source 1..2367: mol_type = other DNA
  CDS 1..2367: protein_id = 72; translation = MNKNNTKLSTRALCSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctgtgcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 113 - DNA, 2367 bp, synthetic construct
  source 1..2367: mol_type = other DNA
  CDS 1..2367: protein_id = 73; transl_table = 1; translation = MNKNNTKLSTRALVSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctggtgagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 114 - DNA, 2367 bp, synthetic construct
  source 1..2367: mol_type = other DNA
  CDS 1..2367: protein_id = 74; transl_table = 1; translation = MNKNNTKLSTRALASFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctggccagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 115 - DNA, 2367 bp, synthetic construct
  source 1..2367: mol_type = other DNA
  CDS 1..2367: protein_id = 75; transl_table = 1; translation = MNKNNTKLSTRALDSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctggacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 116 - DNA, 2367 bp, synthetic construct
  source 1..2367: mol_type = other DNA
  CDS 1..2367: protein_id = 76; transl_table = 1; translation = MNKNNTKLSTRALESFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctggagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 117 - DNA, 2367 bp, synthetic construct
  source 1..2367: mol_type = other DNA
  CDS 1..2367: protein_id = 77; transl_table = 1; translation = MNKNNTKLSTRALFSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctgttcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 118 - DNA, 2367 bp, synthetic construct
  source 1..2367: mol_type = other DNA
  CDS 1..2367: protein_id = 78; transl_table = 1; translation = MNKNNTKLSTRALHSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctgcacagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 119 - DNA, 2367 bp, synthetic construct
  source 1..2367: mol_type = other DNA
  CDS 1..2367: protein_id = 79; transl_table = 1; translation = MNKNNTKLSTRALISFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctgatcagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag

SEQ ID NO: 120 - DNA, 2367 bp, synthetic construct
  source 1..2367: mol_type = other DNA
  CDS 1..2367: protein_id = 80; transl_table = 1; translation = MNKNNTKLSTRALKSFIDYFNGIYGFATGIKDIMNMIFKTDTGG DLTLDEILKNQQLLNDISGKLDGVNGSLNDLIAQGNLNTELSKEILKIANEQNQVLND VNNKLDAINTMLRVYLPKITSMLSDVIKQNYALSLQIEYLSKQLQEISDKLDIINVNV LINSTLTEITPAYQRIKYVNEKFEELTFATETSSKVKKDGSPADILDELTELTELAKS VTKNDVDGFEFYLNTFHDVMVGNNLFGRSALKTASELITKENVKTSGSEVGNVYNFLI VLTALQAQAFLTLTTCRKLLGLADIDYTSIMNEHLNKEKEEFRVNILPTLSNTFSNPN YAKVKGSDEDAKMIVEAKPGHALIGFEISNDSITVLKVYEAKLKQNYQVDKDSLSEVI YGDMDKLLCPDQSEQIYYTNNIVFPNEYVITKIDFTKKMKTLRYEVTANFYDSSTGEI DLNKKKVESSEAEYRTLSANDDGVYMPLGVISETFLTPINGFGLQADENSRLITLTCK SYLRELLLATDLSNKETKLIVPPSGFISNIVENGSIEEDNLEPWKANNKNAYVDHTGG VNGTKALYVHKDGGISQFIGDKLKPKTEYVIQYTVKGKPSIHLKDENTGYIHYEDTNN NLEDYQTINKRFTTGTDLKGVYLILKSQNGDEAWGDNFIILEISPSEKLLSPELINTN NWTSTGSTNISGNTLTLYQGGRGILKQNLQLDSFSTYRVYFSVSGDANVRIRNSREVL FEKRYMSGAKDVSEMFTTKFEKDNFYIELSQGNNLYGGPIVHFYDVSIK
atgaacaagaacaacacgaagctctccacccgcgccctgaagagcttcatcgactacttcaacggcatctacggcttcgcgaccggcatcaaggacatcatgaacatgatcttcaagacggacacgggcggcgacctgacgctcgacgagatcctcaagaaccagcagctcctgaacgacatctcgggcaagctggacggcgtgaacggctccctgaacgacctcatcgcccagggcaacctcaacaccgagctgagcaaggagatcctcaagatcgcgaacgagcagaaccaggtgctgaacgacgtcaacaacaagctcgacgccatcaacaccatgctccgcgtgtacctgcccaagatcacgtccatgctcagcgacgtcatcaagcagaactacgccctgtccctccagatcgagtacctcagcaagcagctgcaggagatctccgacaagctggacatcatcaacgtgaacgtcctcatcaactccaccctgacggagatcaccccggcgtaccagcgcatcaagtatgtgaacgagaagttcgaggagctgacgttcgccaccgagacgtccagcaaggtcaagaaggacggcagccccgccgacatcctggacgagctgaccgagctgacggagctggcgaagtccgtgaccaagaacgacgtcgacggcttcgagttctacctgaacacgttccacgacgtgatggtcggcaacaacctcttcggcaggtcggctctgaagaccgccagcgagctgatcaccaaggagaacgtgaagacgtccggcagcgaagtgggcaacgtctacaacttcctgatcgtcctcacggccctgcaggctcaggcgttcctgaccctgacgacctgccgcaagctcctgggcctcgctgacatcgactacaccagcatcatgaacgagcacctgaacaaggagaaggaggagttcagggtgaacatcctcccgaccctgtccaacacgttcagcaaccccaactacgccaaggtgaagggctccgacgaggacgcgaagatgatcgtggaggctaagcccggccacgccctcatcggcttcgagatctccaacgacagcatcaccgtgctgaaggtctacgaggcgaagctcaagcagaactaccaggtggacaaggactccctgagcgaggtcatctacggcgacatggacaagctcctgtgcccggaccagagcgagcagatctactacaccaacaacatcgtgttccccaacgagtatgtgatcaccaagatcgacttcacgaagaagatgaagaccctccgctacgaggtgaccgccaacttctacgactccagcacgggcgagatcgacctcaacaagaagaaggtcgagtccagcgaggccgagtacaggaccctgagcgcgaacgacgacggcgtgtacatgccgctgggcgtcatctccgagacgttcctcacccccatcaacggcttcggcctgcaggccgacgagaacagccgcctcatcaccctgacgtgcaagtcctacctcagggagctgctgctcgccaccgacctgagcaacaaggagacgaagctcatcgtgccgccctccggcttcatcagcaacatcgtcgagaacggctccatcgaggaggacaacctggagccctggaaggccaacaacaagaacgcctacgtggaccacaccggcggcgtcaacggcacgaaggcgctctacgtgcacaaggacggtggcatcagccagttcatcggcgacaagctgaagccgaagaccgagtatgtgatccagtacacggtcaagggcaagccctccatccacctgaaggacgagaacaccggctacatccactacgaggacacgaacaacaacctcgaggactaccagaccatcaacaagcgcttcaccacgggcacggacctgaagggcgtctacctgatcctcaagtcccagaacggcgacgaggcgtggggcgacaacttcatcatcctcgagatctcgccctcggagaagctgctcagcccggagctgatcaacaccaacaactggacctccacgggcagcacgaacatctccggcaacacgctgaccctctaccagggcggcaggggcatcctcaagcagaacctgcagctcgactccttcagcacctacagggtgtacttctccgtcagcggcgacgcgaacgtgcgcatcaggaactcccgcgaggtcctgttcgagaagaggtacatgtccggcgccaaggacgtgagcgagatgttcaccacgaagttcgagaaggacaacttctacatcgagctgagccagggcaacaacctctacggtggcccgatcgtgcacttctacgacgtctccatcaag